SEQUENCE LISTING (1 sequence)

SEQ ID NO: 1 - protein, 12 aa, Artificial Sequence
  HPC4 tag for C-terminally tagging of alpha-1- antitrypsin
EDQVDPRLIDGK

(19) United States
(12) Patent Application Publication
(10) Pub. No.: US 2023/0099373 A1
(43) Pub. Date: Mar. 30, 2023

(54) METHOD OF DESIGNING CARBOHYDRATES

(71) Applicant: THE REGENTS OF THE UNIVERSITY OF CALIFORNIA, Oakland, CA (US)

(72) Inventors: Nathan E. Lewis, San Diego, CA (US); Wan-Tien Chiang, San Diego, CA (US); Chenguang Liang, San Diego, CA (US)

(21) Appl. No.: 17/777,922

(22) PCT Filed: Nov. 19, 2020
(86) PCT No.: PCT/EP2020/082713

§ 371 Date: May 18, 2022

Related U.S. Application Data
(60) Provisional application No. 62/937,932, filed on Nov. 20, 2019.

(51) Int. Cl. C12N 15/10

(52) U.S. Cl. CPC C12N 15/1058; C12N 15/1082

(57) ABSTRACT

Glycosylated biopharmaceuticals are important in the global pharmaceutical market. Despite the importance of their glycan structures, our limited knowledge of the glycosylation machinery still hinders controllability of this critical quality attribute. To facilitate discovery of glycosyltransferase specificity and predict glycoengineering efforts, here we extend an approach to model biosynthetic pathways for all measured glycans, and the Markov chain modeling is used to learn glycosyltransferase isoform activities and predict glycosylation following glycosyltransferase knock-in/knockout. We apply our methodology to four different glycoengineered therapeutics (i.e., Rituximab, erythropoietin, Enbrel, and alpha-1 antitrypsin) produced in CHO cells, along with o-glycosylation and lipid profiles. Our models accurately predict N-linked glycosylation following glycoengineering and further quantified the impact of glycosyltransferase mutations on reactions catalyzed by other glycosyltransferases. By applying these learned GT-GT interaction rules identified from single glycosyltransferase mutants, our model further predicts the outcome of multi-gene glycosyltransferase mutations on the diverse biotherapeutics. We further apply this to study differential O-glycosylation and lipidomics. Thus, this modeling approach enables rational glycoengineering and the elucidation of relationships between glycosyltransferases and other enzyme classes, thereby facilitating biopharmaceutical research and aiding the broader study of glycosylation to elucidate the genetic basis of complex changes in glycosylation and the lipidome.

### CROSS REFERENCE TO RELATED APPLICATIONS

This application is a U.S. National Stage of International Application No. PCT/EP2020/082713, filed Nov. 19, 2020, which claims the benefit of U.S. Provisional Patent Application No. 62/937,932, filed Nov. 20, 2019, the entire contents of each of which are fully incorporated herein by reference.

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “57777_Seqlisting.txt.” The Sequence Listing was created on May 13, 2022, and is 557 bytes in size. The subject matter of the Sequence Listing is incorporated by reference herein in its entirety.

### TECHNICAL FIELD

The present invention relates to a method of designing carbohydrates.

### BACKGROUND

Glycans are major post-translational modifications, and their structures can directly impact protein characteristics such as binding kinetics, stability, and bioavailability [1, 2]. Therefore, an understanding of their associated biosynthetic pathways is essential for efforts to modify or engineer glycosylation [3-5]. However, since glycan synthesis is highly stochastic and compartmentalized, real-time observation of the glycosylation process is extremely difficult and further complicated by the dynamic structures of the endoplasmic reticulum and Golgi apparatus [6, 7]. Thus it has been challenging to fully understand the dynamic process of glycan synthesis [8]. Given our incomplete understanding of the glycosylation machinery and the costly and laborious glycomics procedures, predictive computational glycosylation models can be invaluable for capturing the features of the complex glycosylation machinery and to understand how the glycosylation machinery responds to external or internal signals and perturbations.

Over the past two decades, several computational models have been built to quantify and model glycan synthesis [9-14]. Recently, a Markov chain method [15, 16] was developed for modeling N-linked glycosylation. This approach has the advantage of being a low-parameter framework that does not require kinetic characterization a priori. The Markov chain process effectively captures the sequential and stochastic nature of glycan modification. In the model, each node represents a glycan and the state transitions are the reactions that add a single sugar to the glycan. Thus, the edge weight is a transition probability, which represents the ratio of total flux making a single glycan from a single precursor glycan, divided by the total flux to make all glycans from that same precursor. The stationary distribution of a Markov model represents the distribution of all fluxes used to make all measured glycans. One can learn the transition probabilities for each reaction by fitting the model to a single glycoprofile, and subsequently predict changes in glycosylation following glycoengineering. Initial studies have laid the groundwork for this approach, but further work is needed to develop models that are broadly applicable and practical to predict the glycosylation outcome of complex glycoengineering for diverse protein products.

One challenge in model-based glycoengineering is how to account for complex regulatory mechanisms of the glycosylation machinery and accurately define enzyme and isozyme specificity for different glycan substrates. Indeed, glycosyltransferase (GT) isozyme specificity and interactions between glycosyltransferases remain unclear and therefore difficult to model. Recently, studies have confirmed functional interactions among several GT isozymes, wherein one GT impacts the function of another. Examples include interactions between β-1,4-galactosyltransferase (B4galt) and Mannosyl-glycoprotein N-acetylglucosaminyltransferases (Mgat), B4galt and β-1,3-N-acetylglucosaminyltransferase (B3gnt), Mgat and B3gnt, and B4galt and beta-galactoside alpha-2,3-sialyltransferase (St3gal) [17-20]. Evidence of these interactions has been based on an observed dependency of glycoprofiles or omics data of GT-knockout cell lines (e.g. ST3GAL1 and B4GALT1 interaction [18]). While these findings suggested GT isozymes interact with each other through direct protein-protein interactions or transcriptional regulation, the specific mechanisms of these interactions and the extent of such interactions have not been extensively studied.

Another significant hurdle for predictive modeling for glycoengineering is our incomplete understanding of GT catalytic specificity. Some glycosyltransferase isozymes, such as those from the B4galt and St3gal families, have more specific catalytic activity on different branches of N glycans [17, 21-24]. However, the complex GT-GT interactions, unknown glycan substrate specificities, and the difficulty in obtaining comprehensive omics and enzyme kinetic data, have all presented great challenges to rational model-driven glycoengineering. Therefore, while considerable efforts have been made for predicting glycosylation patterns of recombinant proteins upon the glycoengineered CHO cells [15, 16, 25, 26], model-based prediction of a glycoengineered glycoprofile from the wildtype glycoprofile is still challenging.

### SUMMARY OF THE INVENTION

The present invention provides a method for determining the biosynthetic basis of a glycosylation pattern or lipid pattern on a cell, glycolipid, tissue, or a protein to be produced by a cell, or an organism to be engineered, comprising: quantifying the impact on the abundance of a glycan or lipid, stemming from enzyme mutation, gene/protein expression changes, or activities of other enzymes to learn enzyme specificity and enzyme interaction rules; and applying learned enzyme specificity and enzyme interaction rules for glycosylation pattern or lipid pattern to predict an outcome of enzyme mutations or gene/protein expression changes on the glycosylation or lipid pattern on a studied protein, lipid, or cell.

In some embodiments the enzyme is a glycosyltransferase (GT) or a glycosidase or an enzyme in lipid biosynthesis or lipid degradation, such as any one enzyme selected from table 1 or table 3.

In some embodiments the enzyme mutations occur by natural mutations, such as by genetic variations of the enzymes or non-naturally by modification of the gene sequence or post-translational modification or enzyme activity through cell culture or chemical treatment, or by changing gene/protein expression levels by natural or non-natural means. In some embodiments the mutations or gene/protein expression changes occur on a single enzyme or multiple enzymes.

In some embodiments the lipids or glycans are free or are attached to a protein, lipid, tissue, recombinant vaccine, or a cell.

In some embodiments the biological source of the glycosylation pattern or lipid pattern (e.g., protein, lipid, cell, tissue sample) is either the same product or a different product from the control product.

In some embodiments the method utilizes a Markov model.

In some embodiments the method to quantify enzyme mutational effects on reactions catalyzed by other enzymes utilizes Markov transition probabilities.

In some embodiments the enzyme mutations or gene/protein expression changes are in different enzymes and/or isozymes.

In some embodiments the cell is a eukaryotic cell, such as a Chinese hamster ovary cell or a human cell, or cancer cell, or mammalian cell, or fish cell, or plant cell, or insect cell, or yeast cell, or fungus, or other microbe.

In some embodiments the glycosylation is any kind of glycosylation, such as N-linked glycosylation, O-linked glycosylation, or glycolipid.

In some embodiments the tissue is any kind of tissue, such as skin, pyloric caeca, or proximal intestine.

In some embodiments the organism is any kind of species, such as a microbe, such as a virus or a bacteria, a plant, or an animal, such as a fish or a mammal.

The present invention provides in a further aspect, a method of producing a protein or lipid or cell or tissue or organism having a desired lipid or glycosylation pattern comprising determining a glycosylation pattern by the methods of the invention, and producing the glycosylated protein or lipid or cell.

The present invention provides in a further aspect a glycan or lipid that is free or part of a protein or cell or tissue or organism produced by the method of the invention.

The present invention provides in a further aspect a method of a glycosylated protein or lipid production or tissue engineering or organism engineering in a biological sample according to the invention, wherein the method is conducted in a biopharmaceutical manufacturing facility.

The present invention provides in a further aspect a method of treatment for a biological sample in need comprising administering to the subject a treatment effective amount of the glycosylated protein or lipid or cell or tissue or organism of the invention, wherein the biological sample is a cell culture.

The present invention provides in a further aspect a method of treatment for a biological sample in need comprising administering to the subject a treatment effective amount of the glycosylated protein or lipid or cell of the invention, wherein the biological sample comprises mammalian cell, such as (e.g., CHO cells or a human subject), or an animal cell, such as (e.g., salmon cells or fish subjects), or a plant cell, or an insect cell, or yeast cell, or fungus, or other microbe.

The disclosure further provides a method for determining a glycosylation pattern on a protein to be produced by a cell or a lipid pattern produced by a cell, comprising: quantifying glycosyltransferase (GT) or lipid enzyme mutations effects on reactions catalyzed by other GTs or lipid enzymes from a glycosylated product or lipid-comprising product to establish learned GT-GT interaction rules or enzyme-enzyme interaction rules, and applying learned GT-GT or enzyme-enzyme interaction rules from the quantifying step to predict an outcome of GT or enzyme mutations on the glycosylation pattern on the protein or lipid pattern.

In embodiments, the disclosure provides a method for determining a glycosylation pattern on a protein or lipid pattern to be produced by a cell, wherein the mutations occur on more than one GT gene or lipid metabolism gene.

In embodiments, the disclosure provides a method for determining a glycosylation pattern on a protein to be produced by a cell, wherein the glycosylated product is a protein.

In embodiments, the disclosure provides a method for determining a glycosylation pattern on a protein or a lipid pattern to be produced by a cell, wherein the protein is different from the glycosylated product or the lipid product and wherein the method utilizes a Markov model.

In embodiments, the disclosure provides a method for determining a glycosylation pattern on a protein or lipid pattern to be produced by a cell, wherein GT or enzyme mutations are in different GT or lipid metabolism isozymes and wherein the cell is a human cell or a CHO cell or a mammalian cell.

In embodiments, the disclosure provides a method of producing a protein having a desired glycosylated pattern comprising determining a glycosylation pattern and producing the glycosylated protein.

In embodiments, the disclosure provides a method of treatment for a subject in need comprising administering to the subject a treatment effective amount of the glycosylated protein.

### BRIEF DESCRIPTION OF THE DRAWINGS

FIG. 1 shows glycoprofiles are fit to the Markov model using global optimization with the Pattern Search algorithm. (Start) A list of all possible reactions (including compartment transportation of glycans) involved in the reaction network is generated based on reaction rules from Table 1. The network complexity is restricted by the number of steps required to generate the most complex glycoform in the WT profile. Transition probabilities (TPs) for each enzyme are assigned to each relevant reaction. (Step 1) Given the assigned TPs, an adjacency matrix of transition probabilities (TPX) is constructed to represent the Markov chain process. (Step 2) Given the TPX and a starting flux feeding into the root node (representing the initial glycan Man₉GlcNAc₂), the predicted glycoprofile is calculated by running the Markov chain model until reaching a stationary flux distribution. (Step 3) The Pattern Search algorithm is used to identify the optimal TP vector by minimizing the RMSE between the predicted glycoprofile and the experimentally measured glycoprofile. The blue dot represents the current TP vector (i.e., polling center), which produced the minimal RMSE=1.3 from all previous rounds of optimization. The newly selected TP vector (red dot) was identified as the optimal solution (the minimal RMSE=0.3) for the next round of optimization. (Step 4) The optimization process will be iterated from (Step 1) to (Step 4) for 1,000 iterations or if less than 1e-6 RMSE reduction is achieved for 50 consecutive runs within 2 hours. Otherwise, the current round of optimization will be discarded. The resulting optimized TP vectors will be used for further analysis. RMSE: root mean squared error.

FIGS. 2A-2C show a prediction performance of the N-glycosylation Markov model. FIG. 2A shows the RMSE and coverage were quantified for the model predictions of EPO produced in glycoengineered CHO cells. We tested three different categories of models: the branch-specific models, the branch-general models, and the random models (i.e., the branch-specific models assigned with random TP vectors). A red star indicates a significant difference of RMSE between the branch-specific and the branch-general models (highest density interval (HDI)=95%). RMSEs of all models (branch-specific models and branch-general models) were significantly different from those of the random models. FIG. 2B shows the glycoprofile of EPO from the CHO cell line wherein B3gnt2 and Mgat4a/4b/5 were all knocked out has the greatest improvement in prediction (RMSE decreased) after introducing branch specificity reactions. FIG. 2C shows the glycoprofile from the B4galt1 knockout showed the least improvement in prediction (RMSE slightly increased and coverage slightly decreased) after introducing branch-specific reactions.

FIGS. 3A-3B show the branch-specific model effectively predicted glycoprofile and optimized transition probabilities for EPO produced in the wild-type CHO cell line. FIG. 3A shows the model-predicted and experimental glycoprofiles of EPO produced in wild type CHO cells. Note that the top ten glycans presented here account for >85% of the total detected glycan abundance in the experimental glycoprofile. FIG. 3B shows the optimized transition probabilities (TPs) by reaction types, in which the TPs were normalized by transport TPs to the next compartments. For example, TPs of reactions localized in cis (Golgi apparatus) were normalized by the TP of glycan transport from cis to medial. The reaction types were separated into three subplots by compartments: cis (cis to medial transport, cg2mg), medial (medial to trans transport, mg2tg), and trans (secretion, tg2ab).

FIGS. 4A-4B show that the branch-specific model identifies indirect effects one GT may have on the activity of other GTs. FIG. 4A shows the Mgat family GTs. FIG. 4B shows the B3gnt family GTs. In each enzyme family, there are three subplots. (i) When a GT isozyme is knocked out (left), we detect changes in the flux of reactions (right), where expected reaction changes are in red, and genetic interactions with other GTs are shown in black. (ii) The heatmap shows the loge fold change transition probability (TP) in for the GT knockout models, compared to the WT models. (iii) The heatmap shows the loge fold change in flux for the GT knockout models in comparison with the WT models. The yellow dots indicate significant non-zero fold changes of the corresponding TP (HDI=95%). The yellow stars indicate the major isozymes whose knockouts resulted in many significantly impacted reactions. The color for the solid line represents the type of reaction impacted by the GT knockout: ‘red’ (GT specific impact) and ‘black’ (GT-GT interaction). The terminal symbol for a line represents the interaction type impacted by the GT knockout: ‘arrow’ denotes activation and ‘filled circle’ is inhibition.

FIGS. 5A-5C show models predict glycoprofiles of multi-GT knockouts. FIG. 5A shows the multiple GT knockout models were built by combining TP vectors from single GT knockout models. We simulated the glycoprofiles for several multi-GT KOs involving the B4galt- and St3gal-family GTs: FIG. 5B shows B4galt1/3 and FIG. 5C shows St3gal3/4 (see Supplementary Figure E1 and E2 in Appendix E for more multi-GT KOs). The relative intensity (m/z) of glycans shown in each barplot correspond to the most abundant 7-10 glycans detected in the corresponding experimental glycoprofiles. For each profile, these m/z values collectively capture >85% of the total experimental signal intensity. Three different heatmaps (from left to right) show the fold change (FC) for TP values: the FC(TP) for single GT KOs, the FC(TP) for multiple GT KOs, and the FC(Flux) for multiple GT KOs. In the multiple GT KOs (FC(TP) and FC(Flux)). The yellow dots indicate significant non-zero fold changes of the corresponding TP or Flux (HDI=95%). Note, EPO is a Human erythropoietin NMR structure from PDB database (PDB ID code: 1buy). Cosine similarity (CosSim) is a nonparametric method used to measure how similar the two vectors (predict and fitted) are. Specifically, it measures the cosine of the angle between two vectors, and the smaller angle means the higher similarity.

FIGS. 6A-6C show multiple GT knockout glycoprofiles can be predicted de novo for diverse drugs. FIG. 6A shows we established a workflow for de novo model prediction of glycoengineered glycoprofile for drugs, wherein TPs learned from glycoengineered EPO (FIG. 5A) are used to inform changes from WT TPs for any engineered glycoprotein. The multiple GT knockout glycoprofiles for FIG. 6B show Enbrel and FIG. 6C shows alpha-1 antitrypsin were predicted directly from their corresponding wildtype models by adjusting the TP vector fold changes (isozyme impact) inferred from the EPO models. For Enbrel and Rituximab, the glycoprofiles with Sppl3 single knockout were treated as the wildtype glycoprofile, as it was the base genotype used prior to GT knockouts.

FIG. 7 shows the O-glycosylation model effectively predicted glycoprofile for mucin-type glycans produced in the skin of salmon samples.

FIG. 8 shows the O-glycosylation model effectively predicted optimized transition probabilities for mucin-type glycan produced in the skin of salmon samples.

FIG. 9 shows the basic setup of fitting the lipidomic Markov models with lipidomic data. An alternative fitting process was described by Liang & Chiang et al (Liang & Chiang et al, 2020).

FIG. 10 shows the current scope of the modular Markov model for lipid metabolism, including syntheses of common human fatty acid (saturated/monounsaturated/polyunsaturated), glycerophospholipids & glycerolipids, sterols, eicosanoids, and sphingolipids.

FIG. 11 shows the fitted lipidomic profile vs. the experimentally measured profile (top 129 signals). Notice that the discrepancy between the measured and the fitted cholesterol values is likely due to the fact that dietary cholesterol is usually abundant and contributes significantly to the plasma level beyond de novo synthesis.

### DETAILED DESCRIPTION

The present invention provides an extensive Markov modeling framework for glycosylation and lipids. Specifically, this modeling framework can learn glycosyltransferase or lipid enzyme activities, including substrate specificities of individual GT or lipid-metabolism isozymes. We further present here a model that predicts the glycosylation of protein drugs produced by glycoengineered Chinese hamster ovary (CHO) cells with multiple glycosyltransferase isozyme knockouts. We demonstrate that our model can estimate the isozyme specificity. We further employed the model to predict the glycoprofiles of multiple glycosyltransferase knockouts. Finally, we show our model effectively predicts glycoengineered glycoprofiles for three diverse recombinant proteins based solely on the wildtype glycoprofiles for three protein drugs (Rituximab, Enbrel, and alpha-1 antitrypsin) produced by CHO cells. These results demonstrate that our updated modeling framework provides a valuable approach for rational glycoengineering and for elucidating the relationships among glycosyltransferases, wherein one can discover the genetic basis of complex glycosylation regulatory mechanisms.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to a pharmaceutically acceptable composition, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

Numbered embodiments of the invention:

1. A method for determining a glycosylation pattern on a protein to be produced by a cell, comprising:

a. quantifying glycosyltransferase (GT) mutations effects on reactions catalyzed by other GTs from a glycosylated product to establish learned GT-GT interaction rules, and

b. applying learned GT-GT interaction rules from the quantifying step to predict an outcome of GT mutations on the glycosylation pattern on the protein.

2. The method of Embodiment 1, wherein the mutations occur on more than one GT gene.

3. The method of Embodiment 1, wherein the glycosylated product is a protein.

4. The method of Embodiment 1, wherein the protein is different from the glycosylated product.

5. The method of Embodiment 1, wherein the method utilizes a Markov model.

6. The method of Embodiment 1, wherein GT mutations are in different GT isozymes.

7. The method of Embodiment 1, wherein the cell is a human cell.

8. A method of producing a protein having a desired glycosylated pattern comprising determining a glycosylation pattern by the method of Embodiments 1-7 and producing the glycosylated protein.

9. A glycosylated protein produced by the method of Embodiment 8.

10. A method of treatment for a subject in need comprising administering to the subject a treatment effective amount of the glycosylated protein of Embodiment 9.

### EXAMPLES

### Example 1

A branch-Specific N-glycosylation Markov Model Effectively Predicts Glycosylation of Glycoengineered CHO Cells

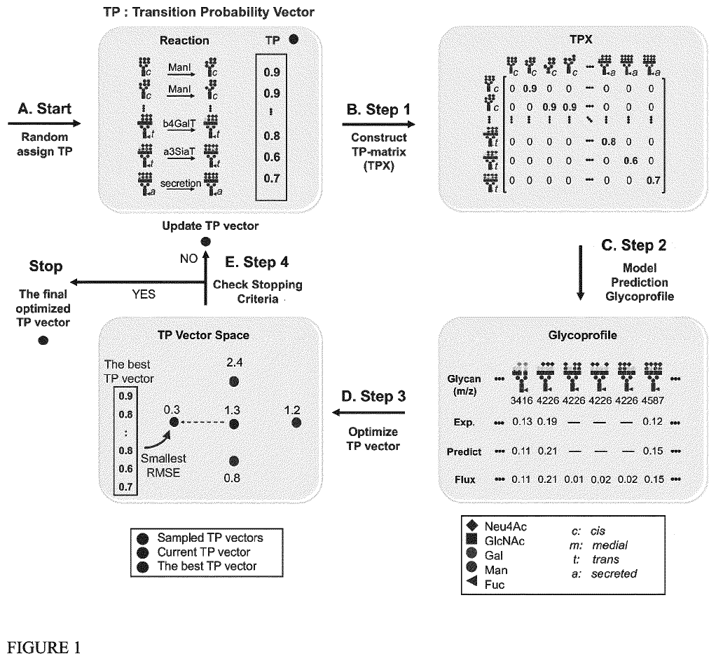

Here, we present four major changes to the N-glycosylation Markov model [15, 16] to overcome the aforementioned challenges (see details in Materials and Methods). To test these changes, we defined two different types of models: a branch-specific model and a branch-general model. The branch-specific model introduced the possibility of branch-specific substrate specificity for each isozyme catalyzing sialylation, galactosylation, and poly-LAcNAc elongation reactions (see details in Materials and Methods). Meanwhile, the branch-general model does not distinguish the glycan substrate branches. We subsequently tested this updated framework (FIG. 1) on glycoprofiles of erythropoietin (EPO) produced in a panel of glycoengineered Chinese hamster ovary (CHO) cell lines [24]. For each model-predicted glycoprofile, we evaluated the performance of our framework by two criteria (see details in Materials and Methods): 1) the root mean squared error (RMSE) for assessing goodness of fit between the model predicted glycan abundance and the experimentally measured glycan abundance; and 2) the coverage, i.e., how many of the experimentally measured glycans were accurately included in our model predictions.

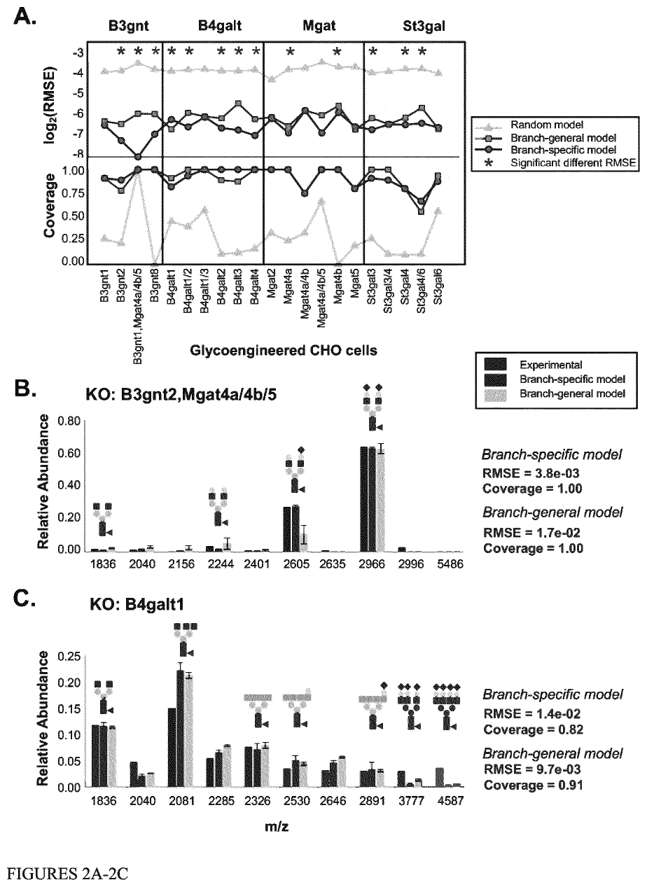

Our newly modified framework demonstrated notable improvements in RMSE and coverage (FIG. 2). Our model improvements included the possibility for enzymes to exhibit specificity to individual branches in a complex N-glycan. While the branch-specific and branch-general models can fit experimental glycoprofiles well (high density interval (HDI)=95%), the branch-specific models provided more accurate results. All model-predicted glycoprofiles have significantly reduced RMSEs in comparison to those produced by random models (i.e., branch-specific Markov models assigned with random TP vectors). In addition, they have high coverage (-90% on average) of experimentally measured glycans. Furthermore, introducing branch specificity significantly enhanced the performance of most model predictions of EPO glycoprofiles from the glycoengineered CHO cells, wherein the B3gnt-, B4galt-, and St3gal-family glycosyltransferases were knocked out. For the most improved glycoprofile (i.e., B3gnt2 and Mgat4a/4b/5 multiple knockouts; FIG. 2B), the branch-specific model produced significantly enhanced performance (RMSE=3.8e-3 and coverage=100%) compared to the branch-general model (RMSE=1.7e-2 and coverage=100%). Even the least improved glycoprofile by the branch-specific model resulted in comparable performance (RMSE=1.4e-2 and coverage=82%) to the branch-general model (RMSE=9.7e-3 and coverage=91%) (B4galt1 knockout; FIG. 2C). However, predictions did not significantly improve with the branch-specific models in the Mgat-family knockout samples; however, this is because the Mgat-family glycosyltransferases (Mgat2, Mgat4a, Mgat4b, and Mgat5) are intrinsically branch-specific in that they are responsible for initiating different branches of N-linked glycans. The improved accuracy after introducing branch specificity was consistent with previous reports wherein individual B4galt and St3gal isozymes differentially contributed to galactosylation and sialylation on different branches [17, 22, 27]. All these results illustrate that the proposed branch-specific framework can more effectively simulate glycosylation of the glycoengineered CHO cells.

Substrate Specificity of Glycosyltransferases can be Predicted by Model Transition Probabilities

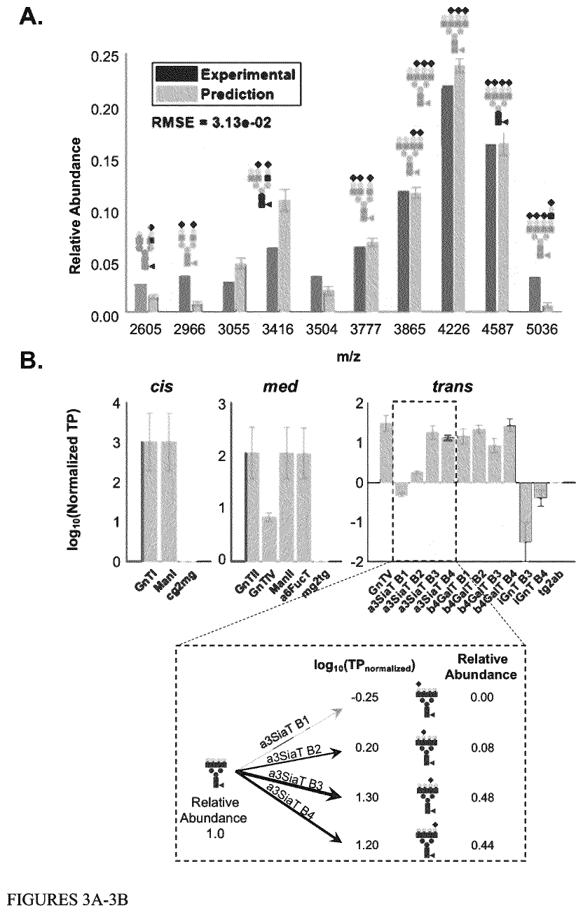

To gain insights into effective glycosylation prediction using the branch-specific models, we closely examined the optimized transition probabilities (TPs) of these models. Each transition probability (TP) is regarded as the probability of transition from one state (substrate) to another (product) for a specific reaction type. The wild-type (WT) model is the basis used to compare with the other glycoengineered models. Therefore, we used the wild-type model to explore if substrate specificity of glycosyltransferases could be described by the TPs. The overall WT model showed a good fit (RMSE=7.72e-03) and complete (100%) coverage (FIG. 3A), which suggested that the modeling framework could effectively account for the experimental glycoprofile.

Four important findings from the model TPs (FIG. 3B) are as follow. First, the TPs of sialylation on branch 3 and 4 (a3SiaT Branch 3-4) were significantly higher than those on branches 1 and 2 (a3SiaT Branch 1-2), which is consistent with the predominant signals of sialylation on branches 3 and 4 from the experimental glycoprofile. This preferential sialylation on branches 3 and 4 compared to branches 1 and 2 has been previously reported [17]. Second, the TPs of branch elongation reactions on branches 3 and 4 (iGnT Branch 3-4) are significantly lower than the TPs of sialylation on branches 1-4 (a3SiaT Branch 1-4). This finding was consistent across all KO profiles. Third, the TPs of GnTII branching were considerably higher than those on GnTIV branching, which was consistent with their differentiated enzyme kinetics [9, 10]. Lastly, glycosyltransferase reactions showed, in general, much larger (tenfold) TPs than intercompartmental transportation TPs in trans Golgi and secretion, with the exception of LacNAc addition. The small TP for LacNAc addition is consistent with its small portion of glycans containing poly-LacNAc in the experimental profile, and previous reports of poly-LacNAc motifs being uncommon in normal mammalian cells [28]. The fitted WT model and the consistency between the TPs and the documented glycosyltransferase activities suggested that the optimized TPs quantitatively describe the substrate preferences collectively contributed by all glycosyltransferase isozymes and shed light on the competition between different glycosyltransferase reactions.

The Branch-Specific Markov Model Reveals Glycosyltransferase Isozyme Specificity and Co-Dependence

Perturbation experiments are widely used to identify potential regulators (e.g., transcriptional regulator), their gene targets, and their regulatory relationships. Here, we employed the same rationale to study how glycosyltransferases regulate N-linked glycan synthesis, using a comprehensive compilation of GT-perturbed glycoprofiles [24]. Specifically, we systematically quantified the contribution of each GT isozyme to different GT reactions by investigating the impact of a single knockout GT on all other reactions. This was done by computing the fold change of TP vectors between the WT model and the GT-knockout models. A significant interaction between a GT and a reaction is detected if the GT knockout significantly altered both the transition probability (TP) and the reaction flux of the GT-knockout model in comparison with those of the WT model (Materials and Methods).

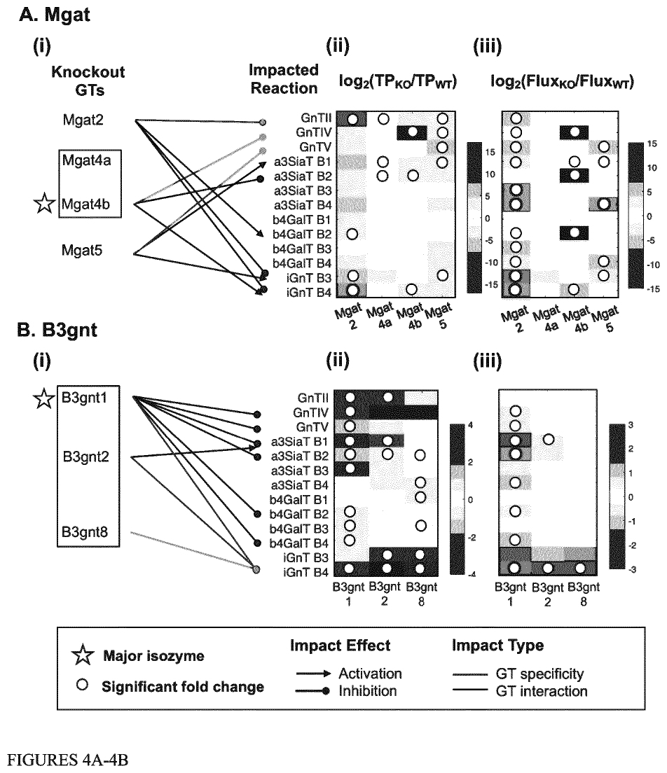

Our results show the total effects of glycosyltransferases on N-linked glycosylation, as identified by the branch-specific models (FIG. 4). Specifically, the loss of function of a glycosyltransferase impacts not only the GT's primary enzymatic function in glycan synthesis, but also the activities of other GTs beyond their own catalytic function. For example, the Mgat-family glycosyltransferases are the key enzymes responsible for the branching of N-linked glycans. We observed that single gene knockout lines for Mgat2, Mgat4b, or Mgat5 gene significantly impacted their own canonical catalyzed reactions—GnTII, GnTIV and GnTV, respectively (see the highlighted red lines in FIG. 4A(i)). Moreover, for the isozymes of Mgat4a and Mgat4b, our model identified Mgat4b as the major isozyme in catalyzing GlcNAc branching. This is consistent with previous observations wherein Mgat4a showed low gene expression levels in CHO cells, and knocking out Mgat4b led to near complete loss of GlcNAc-β1,4-Man-α1,3 branching [24]. Besides their own specifically catalyzed reactions, the model captured the GT interactions between Mgat and other GT isozymes (the black lines in FIG. 4A(i)). We found that Mgat4b or Mgat5 significantly increased the poly-LacNAc extension fluxes, in which the Mgat isozymes seem to compete for the same monosaccharides. Specifically, the Mgat4b KO increases iGnT activity (Branch 4) and the Mgat5 KO increases iGnT (Branch 3). Indeed, following mgat gene knockouts, the Golgi can generate glycans of equivalent mass (or monosaccharide composition) to compensate for the loss of GlcNAc branching by extending the poly-LacNAc [29, 30]. Meanwhile, the lack of GlcNAc branching makes existing branches more accessible to subsequent monosaccharide additions. Another possible explanation could be the redistribution of excessive UDP-GlcNAc from med to trans via inter-cisternal tubules [20]. In addition, the increased sialylation on branch 1 after the Mgat5 knockout was also captured by the model, as reflected by increased free sialyltransferase available to branch 1 following removal of preferentially sialylated branch 4 [31].

The B3gnt-family glycosyltransferases add GlcNAc to the galactose of the N-linked glycans (poly-LacNAc extension). We observed their differentiated catalytic capabilities on LacNAc extension (red lines in FIG. 4B(i)): B3gnt1, B3gnt2 and B3gnt8 single knockout models all carried significantly reduced flux through poly-LacNAc extensions on branch 4. The result was consistent with the fact that they all contribute to poly-LacNAc formation in N-linked glycosylation [20, 32, 33]. Beyond its direct impact on the poly-LacNAc extension, a B3gnt1 knockout also significantly resulted in changes in the reactions of branching (GnTIV/V), galactosylation (b4GalT Branch 2/4), and sialylation (a3SiaT Branch 1/2). The discovery is consistent with the finding that the gene products of B4galt1 and B3gnt1 co-localize and physically associate in vivo [34, 35], and knocking out B3gnt1 will impact B4galt1 activity and all other interacting glycosyltransferases. B3gnt1 knockout further impaired Mgat4 and Mgat5 branching in addition to sialylation on most branches as shown by the modeling result. Last but not least, while knocking out both B3gnt1 or B3gnt8 impacted poly-LacNAc elongation, only knocking out B3gnt2 significantly impacted total poly-LacNAc extension flux, resulting in significantly increased sialylation on branch 1 due to diminished competition for St3gal isozymes.

Intriguingly, despite that glycosylation has been known as a non-templated glycan synthesis process, all these results suggest glycosylation to be a robust cellular process with the mechanism in response to GT knockout. While interactions between different isozymes in the same family and other GTs are complicated, our model TPs and flux variation were highly consistent with the GTs' known interactive mechanisms or enzyme kinetics. While further experimental validation is required, our model captured glycotransferase isozyme specificity and suggested how glycosyltransferases influence the activities with each other. These insights shed light on the regulation of N-linked glycosylation.

Glycoprofiles for Complex GT Mutants can be Predicted from Single GT Knockout Models

Genetic interactions complicate the prediction of multi-gene knockout phenotypes, especially when the genes are involved in the same pathway. However, since our modeling framework captures the pathway architecture in N-linked glycosylation, we examined if our models trained on single GT mutants could predict glycoprofiles for mutants with more complex genotypes. Specifically, after obtaining the fitted models of single GT knockouts, we extracted transition probability (TP) vectors from these models and combined them to create new TP vectors, which predicted the GTs' collective influence on the N-glycosylation synthesis for the combinatory knockout experiments. We developed an algorithm that enabled us to assess the significance of TP fold change vector elements for a multiplex glycoengineered Markov model (Materials and Methods). Briefly, our algorithm identifies the fitted single-knockout TPs that define the changes in reaction flux following the knockout of an isozyme. It subsequently merges these TPs for all gene knockouts in the more complex mutant to establish a new multi-gene knockout TP vector for glycoprofile prediction.

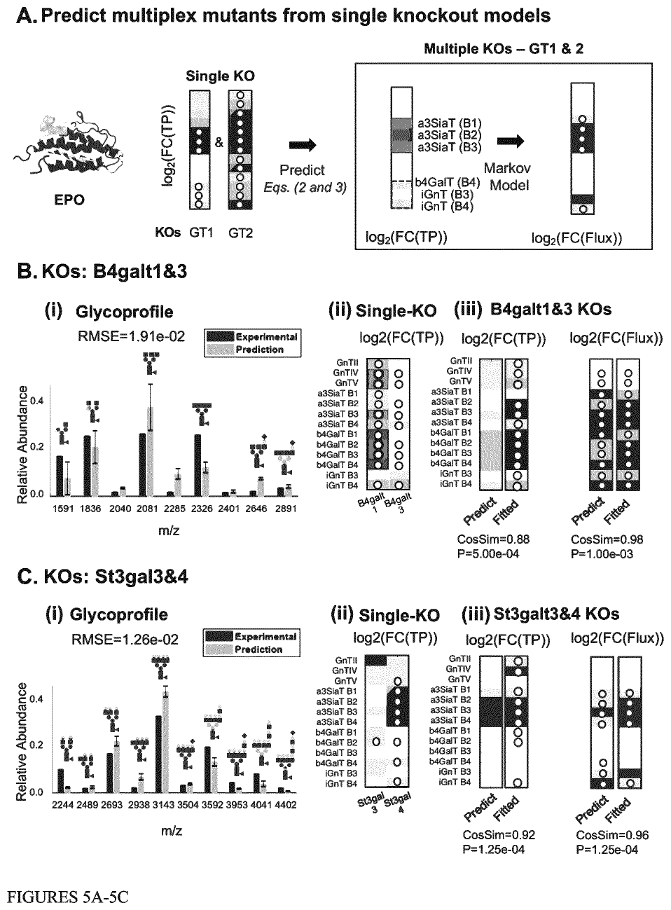

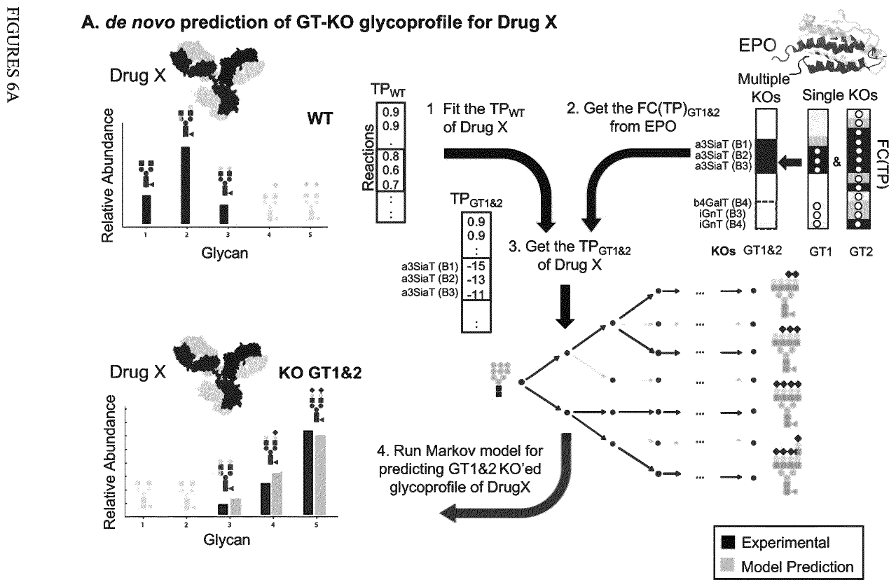

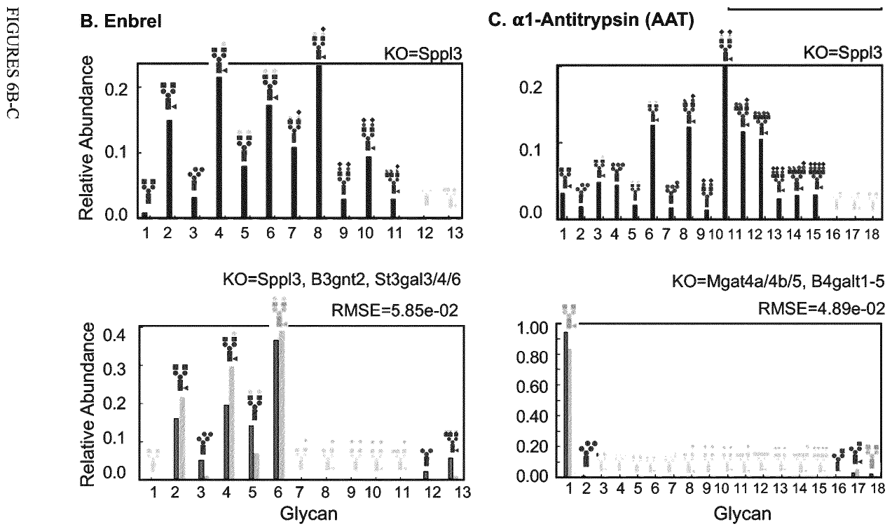

The predicted glycoprofiles produced by our models showed high consistency with the experimental profiles for the multi-gene knockouts. Specifically, glycoprofiles were accurately predicted for eight erythropoietin (EPO) samples, each produced in different glycoengineered CHO cells with different combinations of glycosyltransferases knocked out. The multi-gene knockout models predict glycoprofiles with excellent performance (all log₂(RMSEs)<−5.5), comparable to the fitting performance in general (FIG. 2A). Furthermore, the model reliably predicted glycoprofiles involving major St3gal or B4gal isozyme knockouts, which had remained challenging due to their complicated interactions with the functions of other glycosyltransferases and difficulty in correlating specific isozyme manipulation with model parameters. For example, the double B4galt/St3gal isozyme knockouts (B4galt1/3 and St3gal3/4; FIGS. 5B and 5C) reduced sialylation even further than B4galt1 knockout alone (FIG. 4B), validating the active roles of B4galt2 and B4galt3 in galactosylation despite their lack of impact when they were individually knocked out [17]. The robust prediction performance further validated the quantification of isozymes' catalytic capabilities by TP vectors and alluded to the model's potential for de novo prediction of biologically accurate glycoprofiles for glycoengineered CHO cell lines. Indeed, by comparing the fitted TPs to the predicted TPs, for each isozyme we identified the fluxes they impacted and quantified their influence on those fluxes. Intriguingly, while B4galt2 and B4galt3 only applied small modifications to TPs beyond B4galt1's impact, the predicted glycoprofiles were distinctive from each other and consistent with the fitted results. Therefore, our modeling framework can be used to predict glycoprofiles of multiple glycotransferase knockouts using single GT knockout models.

Glycoprofiles can be Predicted for Additional Glycoengineered Drugs De Novo, Based Solely on TP Fold Changes Learned from EPO

Various factors impact the glycoprofile of each unique protein, including protein sequence, structure, post-translational modifications, etc. Thus, it is unclear if glycosyltransferase preferences for one glycoprotein substrate will translate to other protein substrates. Thus, we tested if the EPO-trained models could be generalized to predict the glycoprofiles of other glycoengineered protein drugs (see details in Materials and Methods) directly from their corresponding wildtype models (see FIG. 6A for procedure). To do this, the modeling framework learns TPs for the wildtype glycoprofiles of a new protein. We hypothesized that the TP fold changes captured by the EPO models are strongly associated with the isozymes' intrinsic catalytic capabilities and are therefore applicable to other protein drugs produced by CHO cells. In particular, N-linked glycosylation for EPO uses a wide variety of glycosyltransferase isozymes from all four families (Mgat-, B4gat-, St3gal-, and B3gnt-family) and produces complex glycoprofiles. This allowed us to extract rich and more complete information regarding the isozyme activities and preferences. Thus, this information enables the prediction of equally or less complex glycoprofiles of other protein drugs, which may only utilize a subset of glycosyltransferase isozymes.

Testing our hypothesis, we predicted glycoprofiles for three different drugs (Rituximab, alpha-1 antitrypsin, and Enbrel) produced by CHO cell lines with both single and multiplex GT knockouts covering all the four GT families (FIGS. 6B-C). We found that the predicted KO glycoprofiles demonstrated outstanding performance (all log₂(RMSE)<−4) for both slightly impacted (Rituximab; Figure F1B) and severely impacted (alpha-1 antitrypsin; FIG. 6C) glycoprofiles, in addition to the highly complex Enbrel glycoprofiles (FIG. 6B and F1A). These results indicated that the transition probabilities from EPO could be used to predict the glycoprofiles of other protein drugs.

The Low-Parameter Markov Framework is Further Simplified for More Efficient Modeling of Glycosylation

Over the past two decades, several mathematical models have provided insights into the complex glycosylation machinery [8, 10, 25, 36, 37]. Here, we extended our low-parameter Markov model framework [15] and demonstrated its ability to predict GT substrate specificity and the outcome of multiplex glycosyltransferase mutations. This low parameter approach does not require the input of kinetic or concentration information, and we further simplified it by updating the transition probability (TP) formulation only describe the activity of the 20 different glycosyltransferases and glycosidases (the previous formulation considered all transitions at each branch point in the biosynthetic network independently). In essence, the updated framework makes strong ties between transition probabilities (TPs) and the enzymes' catalytic capabilities, which is especially effective for modeling glycoengineered glycoprofiles. By closely examining the fluxes of glycosylation models, our results demonstrated that the new method comprehensively captures the active parts of the glycosylation network following glycoengineering. For example, our single knockout models (Mgat4b and Mgat5) identified significantly increased poly-LacNAc extension fluxes, which is consistent with known competition between the Mgat isozymes and B3gnt isozymes for the same GlcNAc monosaccharides ([29, 30], see Results). Furthermore, we replaced the original flux variability analysis (FVA) with the efficient global optimization algorithm—Pattern Search. At present, we are able to model a glycoprofile within 2 hours for a model with 8,435 glycans and 19,719 reactions, which took a few days to complete by using the original FVA optimization algorithm. Both the reduced number of TPs and the new algorithm make the computational time of fitting a large reaction network more practical.

Computational Analyses can Unravel Multi-Glycosyltransferase Interactions Impacting Activities Beyond their Simple Enzyme Rules

A critical challenge in developing a predictive glycosylation model lies in the difficulties of quantifying the genetic interactions beyond each GT's simple enzyme rules. Recently, large amounts of glycoprofiling data were generated from GT knockouts. These data allow us to capture how each perturbed GT impacts the expected activities of other GTs, providing new insights into the genetic interactions between different glycosyltransferases. We presented here a comprehensive documentation of genetic interactions between glycosyltransferases. Importantly, while GTs are expected to be specific toward their own catalytic functions, we show here that knocking out a glycosyltransferase could impact the function of other GTs. For instance, the Mgat2 knockout decreased its own GnTII reaction but promoted the b4GalT—Branch2 reaction (galactosylation). The above findings raise at least two important issues for biotherapeutic glycoengineering applications. The first issue concerns the extent to which potential unintended GT changes (off-target effects) may arise from a specific GT perturbation, and rational glycoengineering of a specific glycoform could be more non-intuitive than we thought. However, as multiplex GT mutants are constructed and profiled, computational approaches as presented here can identify and account for genetic interactions, thus helping improve rational glycoengineering of biotherapeutics. Furthermore, such computational analyses can be leveraged to guide research into the underlying molecular mechanisms (e.g., transcription, epigenetic, and feedback loops) regulating GT-GT interactions.

Predicting Glycosylation with Minimal a Priori Knowledge

One of the major goals for developing glycosylation models is to provide valuable guidance for glycoengineering therapeutic proteins. The present findings of this research contribute to the field's understanding of the underlying rules acting on single GT knockout models resulting in a complex GT mutated model, which enables us to predict glycoprofiles of multi-gene mutations. The excellent performance for our model indicates that TP fold changes capture the specificity of each isozyme. These TP values that were learned and quantified from glycoengineered EPO profiles could be combined to predict the glycoprofiles from multi-gene mutants producing distinct glycoproteins, as long as one has the WT glycoprofile for the new protein of interest. These results lend credence to the hypothesis that the GT interactions are generally encoded in the glycosylation machinery, which could be captured by our glycosylation model. It is apparent that the effect of complex GT knockout strategies impact different biologics in a similar manner. The satisfying accuracy of prediction results and the generalizability of the model pave the way to prospective research for consolidating the study of glycosyltransferase interactions and for rational glycoengineering for better biopharmaceuticals.

Disentangling the Functions of Different Isozymes

We demonstrated here that model-based analyses can discover or reinforce our understanding of the unique functions of different GT isozymes. We found that there are major isozymes whose knockouts impacted more reactions. Several studies have demonstrated the diversity of GT isozymes. For example, in different mammalian cells, Mgat4b is more responsible for the GlcNAc-β1,4-Man-α1,3 branching [24], B4galt1 for galactosylation [24, 38], St3gal4 for sialylation [39], and B3gnt2 for poly-GlcNAc formation [20, 32, 33]. Our glycosylation modeling framework confirmed putative GT specificity but reinforced the dominant role of these major GT isozymes in CHO cells. Furthermore, our results also suggest that different GT isozymes have differences in their functions. For instance, our model suggests that knocking out St3gal6 or St3gal4 had the most severe impact on sialylation (decreased sialylation fluxes by >85%), but knocking out St3gal3 had little influence. These results are in accordance with its primary role for sialylation [39]. This knowledge is particularly important and could be applied to improve product quality through glycoengineering by being able to partially dial down some glycan epitopes. Indeed, sialylation is a key factor in most glycoengineering, since it can improve the serum half life and activity of these drugs [40]. On the other hand, limiting sialylation on monoclonal antibodies (mAb) could enhance antibody-dependent cell-mediated cytotoxicity (ADCC) and complement-dependent cytotoxicity (CDC). In these cases, we could consider knocking out a few sialyltransferases (St3gal3, St3gal4, or St3gal6) for better control of the sialylation on mAb. The proposed model framework thus provides a toolbox that could help identify the best combination of different GT isozymes for desired glycoforms. The more we are able to disentangle the functions of different isozymes, the better we can ultimately control the glycosylation machinery, which should be an important steppingstone toward rational glycoengineering.

Conclusions

Here we present a substantial improvement to the Markov chain modeling framework for glycosylation, which accounts for branch-specificity and isozyme preference. These refined models effectively simulated the N-glycosylation process of recombinant proteins produced by various glycoengineered CHO cell lines. The essence of our model is transition probabilities, which capture the catalytic capabilities of glycosyltransferase isozymes and quantify the changes in glycosylation after knocking out various isozymes. Exploiting the new modeling framework, we systematically examined the potential interactions between different families of glycosyltransferases and their substrate/branch specificities, which provides insights into the roles of GT isozymes in specific contexts. Our results here further demonstrated that we can predict complex glycoengineered glycoprofiles from single-KO models. With the learned fold changes of transition probabilities from EPO, we achieved de novo prediction of GT-KO glycoprofiles directly from their WT glycoprofiles for new protein drugs produced by CHO cells. Therefore, as this framework facilitates rational glycoengineering of various glycosylated protein drugs, it will accelerate the development of effective, safe, and affordable glycosylated biopharmaceuticals.

Materials and Methods

Framework of Markov Chain Model for the N-Linked Glycosylation

The Markov model of glycosylation is implemented as previously published [15], with a few adaptations described here to improve the fitting to glycoprofiles subsequent model predictions (FIG. 1). In essence, this updated Markov model framework can be used for modeling the N-glycosylation process by accounting for all measured and quantified glycans. The new proposed model provides additional capabilities, such as the means to address glycosyltransferase isozyme specificity and interactions for model-based rational glycoengineering. Here, we highlight four major changes in the newly proposed framework to overcome the aforementioned challenges. First, our updated framework enables the use of a complete glycosyltransferase reaction network rather than a tailored one (i.e., we do not trim out unmeasured glycans), which enables us to fit the model with more accurate transition probabilities (TPs) (see details in the Discussion). Second, instead of using the COBRA toolbox [41], we have deployed the Pattern Search algorithm for obtaining the best TP vector. This well-established global optimization algorithm has efficient convergence in a high-dimension solution space [42-46]. Third, instead of optimizing hundreds of transition probabilities in the transition probability matrix (TPX) by using the COBRA framework [15, 47], only the twenty TPs are defined, corresponding to the twenty different reaction types (Table 1), which were optimized by the Pattern Search algorithm. Fourth, the TPs for sialylation, galactosylation, and poly-LacNAc elongation were further distinguished by the branch on which the corresponding monosaccharides were added (Table 1). The reaction rules were compiled and curated for consistency based on previous publications on Markov or kinetic-based models [10, 12, 15, 25, 48, 49]. Notably, unlike all previously published models, the reaction constraint for a6FucT was removed from its reaction rule as new studies have confirmed the feasibility and presence of fucosylation without the presence of α1,3-branch (Branch 1/3) GlcNAc moiety [50-52]. For branch-general models, substrate branches were not distinguished for B4GalT (BX), a3SiaT (BX), and iGnT (BX) (10 reaction types), resulting in only B4GalT, a3SiaT, and iGnT reaction types (3 reaction types). ‘X’ denotes branches numbered 1, 2, 3, or 4, which represent GNb2|Ma3, GNb4|Ma3, GNb2|Ma6, and GNb6|Ma6 respectively.

TABLE 1
Branch-specific reaction rules for the N-linked glycosylation model

| | | | | Locali- |
|---|---|---|---|---|
| Reaction*^{c} | Substrate*^{a} | Product*^{a} | Constraint*^{a, b} | zation |
| ManI | (Ma2Ma | Ma | — | cis |
| ManII | (Ma3(Ma6)Ma6 | (Ma6Ma6 | (GNb2\|Ma3 | medial |
| ManII | (Ma6)Ma6 | (Ma6 | (GNb2\|Ma3 | medial |
| GnTI | (Ma3(Ma3(Ma6)Ma6)Ma4 | (GNb4Ma3(Ma3(Ma6)Ma6)Ma4 | — | cis |
| GnTII | (GNb2\|Ma3(Ma6)Mb4 | (GNb2\|Ma3(GNb2Ma6)Mb4 | — | medial |
| GnTIV | (GNb2Ma3 | (GNb2(GNb4)Ma3 | — | medial |
| GnTV | (GNb2Ma6 | (GNb2(GNb6)Ma6 | — | trans |
| a6FuT | GNb4GN | (GNb4(Fa6)GN | ~* . . . Ma2 | medial |
| b4GalT (B1) | (GN | (Ab4GN | * . . . GNb2\|Ma3 | trans |
| b4GalT (B2) | (GN | (Ab4GN | * . . . GNb4\|Ma3 | trans |
| b4GalT (B3) | (GN | (Ab4GN | * . . . GNb2\|Ma6 | trans |
| b4GalT (B4) | (GN | (Ab4GN | * . . . GNb6\|Ma6 | trans |
| a3SiaT (B1) | (Ab4GN | (NNa3Ab4GN | * . . . GNb2\|Ma3 | trans |
| a3SiaT (B2) | (Ab4GN | (NNa3Ab4GN | * . . . GNb4\|Ma3 | trans |
| a3SiaT (B3) | (Ab4GN | (NNa3Ab4GN | * . . . GNb2\|Ma6 | trans |
| a3SiaT (B4) | (Ab4GN | (NNa3Ab4GN | * . . . GNb6\|Ma6 | trans |
| iGnT (B3) | (Ab4GN | (GNb3Ab4GN | * . . . GNb2\|Ma6 | trans |
| iGnT (B4) | (Ab4GN | (GNb3Ab4GN | * . . . GNb6\|Ma6 | trans |

*^{a}‘A’, ‘F’, ‘GN’, ‘M’, and ‘NN’ represent galactose, fucose, GlcNAc, mannose, and NAcNAc respectively, whereas ‘aX’ or ‘bX’ (where ‘X’ is a number) represents an alpha or beta glycosidic bond connecting the two adjacent sugars (e.g. a3 represents alpha 1,3 glycosidic bond).
*^{b}‘*’ indicates the position of added moiety and associated bond strings, ‘ . . . ’ a string of any length with all brackets matched, and ‘|’ a branching point.
*^{c}‘B1-4’ indicate the four possible branches of a glycan as described by the Constraint
Note that, we specified the glycans as linear code strings with complete linkage and composition information for easy computation in the model.

Model Evaluation Metrics—RMSE and Coverage

Two model evaluation metrics were used for evaluating the performance of our models. The first one is the root mean squared error (RMSE) for assessing the goodness of fit between the model-predicted glycan intensities and the experimentally measured glycan intensities. The experimental glycoprofiles were fit by minimizing the RMSE of TP vectors between the model prediction glycoprofile and the experimental glycoprofile. The RMSE was calculated by equation 1, where N represents the number of all possible glycan compositions (m/z values) in the experimental glycoprofile. y_{pre,i} (y_{exp,i}) represented the predicted (experimentally measured) signal intensity measured at the ith m/z value (glycan composition).

$$RMSE = \sqrt{{\sum }_{i}^{N} \frac{{( {y}_{pre , i} - {y}_{exp , i} )}^{2}}{N}} \qquad ( 1 )$$

Statistical significance was further assessed using the highest density interval (HDI), wherein the statistical meaning of HDI=95% is that the two groups of tested models are significantly different with a 95% confidence interval.

Another model evaluation metric is ‘coverage’ for assessing how many of the experimentally measured glycans were accurately included among the glycans predicted by our framework. For an experimental glycoprofile, the m/z values corresponding to glycans with the top signal intensities and collectively representing at least 90% of the total signal intensity were selected as experimentally detected glycans. The coverage was defined as the ratio of these glycan compositions that can be captured by the glycoprofiles predicted by the Markov models (branch-specific and branch-general models).

Predicting Multiple GT Knockouts from Single GT Knockout Models

The TP vector for a given multiple knockout glycoprofile was derived from the TP vectors of the relevant fitted single-knockout glycoprofiles. Four criteria were used to define the significance of TP vector elements for a multiplex glycoengineered Markov model. Specifically, the fitted single-knockout TPs are required for substantiating the impact of knocking out an isozyme on the reactions listed in Table 2. First, the TP fold change of reaction i after knocking out glycosyltransferase k must be statistically different from 0 (i.e., the 95% highest density interval (HDI) does not include 0 from the BEST analysis. Assessment of the statistical credibility of flux and TP using Bayesian estimation was used. Second, the mean flux fold change of reaction i, after knocking out glycosyltransferase k, must be have a scaling factor of at least 1.5 fold (|log₂(mean flux fold change)|≥0.58), and the mean flux fold change±one standard deviation does not include 1. Then, another two additional criteria were established for predicting a new TP for a glycoprofile with combinatorial glycosyltransferase knockouts. Third, if all isozymes of the same family are knocked out, the TP log₂ fold changes of the associated direct reaction(s) will be reduced to at most −10 (eliminating fluxes of direct reactions). Fourth, log₂(flux fold change) and log₂(TP fold change) must have the same sign for the KO model of glycosyltransferase k. These four criteria were applied in equations 2-3 for deriving the final combined TP vectors:

$${log}_{2} ( FC ( {TP}_{Ci , k} ) ) = {\sum }_{k} {log}_{2} ( FC ( {TP}_{Fi , k} ) ) + \frac{1}{{A}_{i}} {\sum }_{k} {log}_{2} ( FC ( {TP}_{Si , k} ) ) \qquad ( 2 )$$

$$FC ( {TP}_{Fi , k} ) and FC ( {TP}_{Si , k} ) ) = 0 , if any of the four criteria are not met . \qquad ( 3 )$$

Briefly, the fold change of the transition probability values, FC(TP_{Fi,k}), is defined as the TP fold change of reaction i, which is the reaction (denoted as ‘F’) directly catalyzed by GT-isozyme k, and FC(TP_{Si,k}) is the reaction (denoted as ‘S’) potentially impacted by GT-isozyme k knockout. In which, Table 2 listed the reactions directly catalyzed by a given enzyme based on their known reaction rules. The potentially impacted reactions are all the other reactions not directly influenced by the GT-isozyme k knockout, which can be indirectly influenced by either kinetically or through other known interactions (i.e. B4galt and Mgat4). Aᵢ is the number of non-zero FC(TP_{Si,k}), and FC(TP_{Ci,k}) is the TP fold change of reaction i for the predicted multiple glycosyltransferase knockout glycoprofile. FC (Fold change) is defined as the TP of reaction i for the fitted WT divided by the predicted multiple GT-KO glycoprofiles. The derived (predicted) TP vector for a combined GT-KO Markov model was then assigned to the initial TPX, which was used in models to predict the multiple knockout glycoprofile (FIGS. 1B and 1C). Here, the nonparametric cosine similarity is used to measure how similar between two vectors (predict and fitted) for flux and TP. Specifically, it measures the cosine of the angle between two vectors, and the smaller angle means the higher similarity.

TABLE 2
Reactions potentially influenced by
the knockout of a given enzyme.

| Gene Knockout | Direct Reactions* |
|---|---|
| Mgat2 | GnTII |
| Mgat4a/4b | GnTIV |
| Mgat5 | GnTV |
| St3gal3/4/6 | a3SiaT (Branch 1-4) |
| B4galt1/2/3/4 | b4GalT (Branch 1-4) |
| B3gnt1/2/8 | iGnT (Branch 3/4) |

*Direct reaction included reactions directly catalyzed by the given enzyme encoded by the knocked-out gene(s), whereas the potentially impacted (dependent) reactions are those whose TPs may be influenced by the knockout of a given enzyme.

Protein Purification and Glycan Analysis for Additional Glycoengineered Drugs

GT-knockout cell line generation and model protein expression. Glyco gene knockout cells lines were derived from the CHO-S cell line (Gibco Cat. #A11557-01), and they were generated and verified according to the procedures described previously [53]. Cells were cultured in CD CHO medium (Gibco 10743-029) supplemented with 8 mM L-glutamine (Lonza BE17-605F) and 2 mL/L of anti-clumping agent (Gibco 0010057AE) according to the Gibco guidelines. The day prior to transfection, cells were washed and cultured in exponential phase in medium not supplemented with anti-clumping agent. At the day of transfection, viable cell density was adjusted to 800,000 cells/mL in 125 mL shake flasks (Corning 431143) containing 30 mL medium only supplemented with 8 mM L-glutamine. Plasmids encoding for Rituximab, Enbrel, and alpha-1-antitrypsin, respectively, were used for transient transfections. For each transfection, 30 ug plasmid was diluted in OptiPro SFM (Gibco 12309019) to a final volume of 750 uL. Separately, 90 uL FuGene HD reagent (Promega E2311) was diluted in 660 uL OptiPro SFM. The plasmid/OptiPro SFM mixture was added to the FuGENE HD/OptiPro SFM mixture and incubated at room temperature for 5 minutes. The resultant 1.5 mL plasmid/lipid mixture was added dropwise to the cells. Supernatants containing model protein were harvested after 72 h by centrifugation of cell culture at 1,000g for 10 minutes and stored at −80° C. until purification and N-glycan analysis.

Protein purification and N-glycan Rituximab and Enbrel were purified by protein A affinity chromatography. A 5-mL MAbSelect column (GE Healthcare) was equilibrated with 5 column volumes (CV) of 20 mM sodium phosphate, 0.15 M NaCl, pH 7.2. Following column equilibration, the supernatant was loaded, the column was washed with 8 CV of 20 mM sodium phosphate, 0.15 M NaCl, pH 7.2, and the protein was eluted using 0.1 M citrate, pH 3.0. Elution fractions (0.5 mL) were collected in deep-well plates containing 60 μL of 1 M Tris, pH 9 per well. alpha-1-antitrypsin, C-terminally tagged with the HPC4 tag (amino acids EDQVDPRLIDGK), was purified over a 1-mL column of anti-protein C affinity matrix according to the manufacturer's protocol (Roche, cat. no. 11815024001). 1 mM CaCl₂was added to the supernatants, equilibration buffer and wash buffer. The proteins were eluted in 0.5 mL fractions using 5 mM EDTA in the elution buffer. For all four proteins, elution fractions containing the highest concentration of protein were concentrated ten-fold using Amicon Ultra 0.5-mL centrifugal filter units (MWCO 10 kDa). 12 μL of concentrated protein solutions (concentrations varying between 0.1 and 1 mg/mL) were subjected to N-glycan labeling using the GlycoWorks RapiFluor-MS N-Glycan Kit (Waters).

N-glycan analysis. N-glycans were labeled with GlycoWorks RapiFluor-MS N-Glycan Kit (Waters). Briefly, 12 uL concentrated culture supernatant labeled according to the manufacturer's instructions. Labeled N-Glycans were analyzed by LC-MS as described previously [53]. Initial conditions 25% 50 mM ammonium formate buffer 75% Acetonitrile, separation gradient from 30% to 43% buffer. MS were run in positive mode, no source fragmentation. The normalized, relative amount of the N-glycans is calculated from the area under the peak with Thermo Xcalibur software (Thermo Fisher Scientific).

### Example 2

O-Glycosylation Markov Model Effectively Predicts Glycosylation of Salmon Skin Mucus

O-glycosylation plays important roles in developmental and immunological functions in biological systems (Joshi et al., 2018); especially, the mucin-type O-glycosylation has been investigated for its potential use in drug and vaccine development.(Tarp and Clausen, 2007) For example, the pathogen, furunculosis-causing bacterium Aeromonas salmonicida ssp. salmonicida, binds differentially to mucins isolated from skin and intestinal regions of the Atlantic salmon (Padra et al., 2014), resulting in substantial loss in the salmon industry. Therefore, investigation on the O-glycosylation could benefit salmon production, especially to improve the health of the fish and its impact on nutritional value.

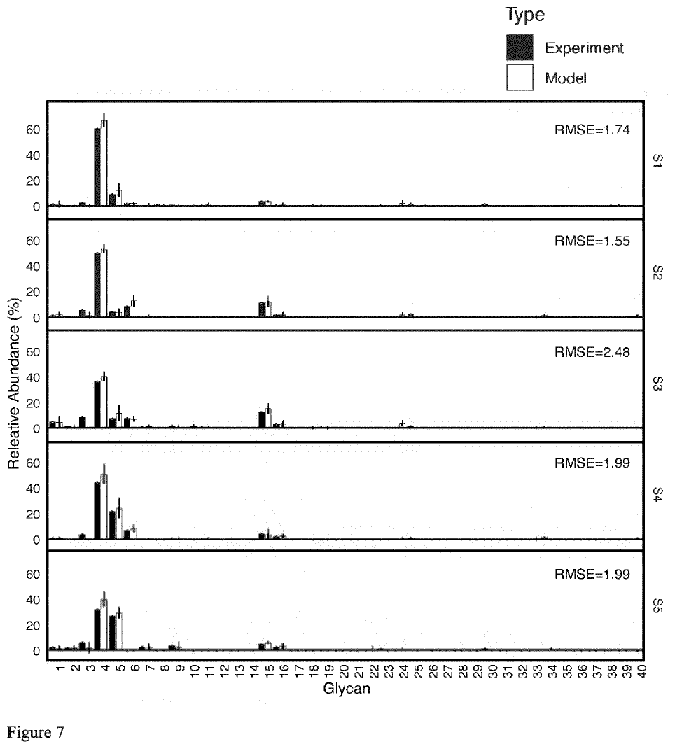

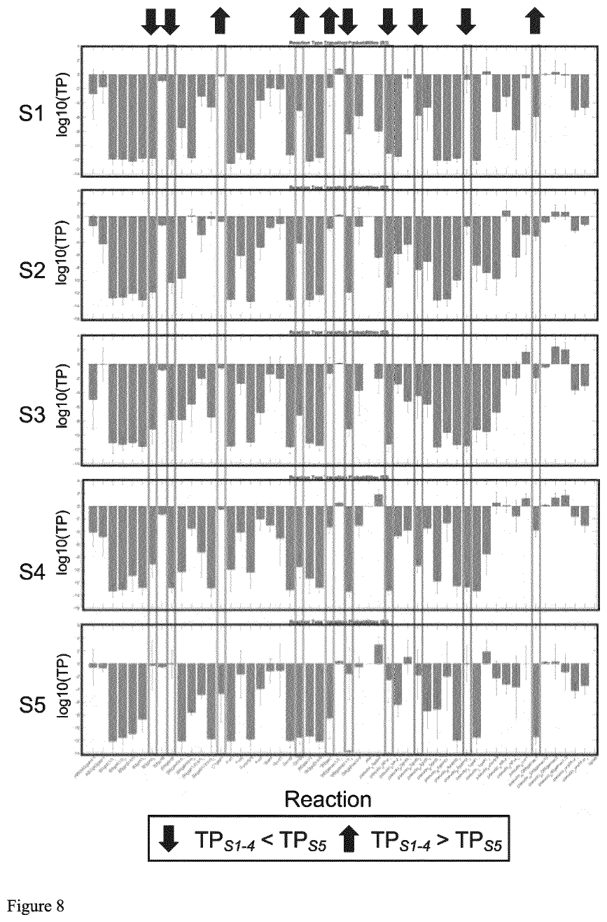

Recently, Jin et al. (Jin et al., 2015) experimentally measured the mucin-type O-glycosylation of Atlantic Salmon. In this study, we developed the first O-glycosylation model on mucin proteins composing the mucus layer of the Atlantic Salmon. Table 1 shows the reaction rules for reconstructing the mucin-type O-glycosylation network. We first fit the reconstructed O-glycosylation network with the experimental data of 5 salmon skin samples (Table 2). Our results demonstrated excellent performance (RMSE ranged between 1.74 to 2.48; average RMSE=1.95) and most of the detected glycans were identified by our model (coverage greater than 95%) for predicting the mucin-type O-glycosylation of salmon skin samples (FIG. 7). Moreover, our model is capable of studying the possibility for enzymes to exhibit specificity to individual branches in a mucin-type O-glycan (FIG. 8). Interestingly, our model showed that the skin sample-S5 might have several enzyme catalyzed reactions differentially expressed from the other skin samples (S1-4). Specifically, we observed 6 reactions (highlighted in the red rectangles) that transition probabilities (TPs) of sample S5 are higher than TPs of samples S1-4. We also observed 4 reactions (highlighted in the green rectangles) that transition probabilities (TPs) of sample S5 are lower than TPs of samples S1-4. These results demonstrated that sample S5 has different O-glycan synthesis capability. Indeed, we observed that sample S5 has lower expression of glycan #4 (relative abundance <40%) but higher expression of glycan #5 (relative abundance >20%) compared with samples S1-4 (relative abundance>40% on glycan #4 expression and relative abundance<20% on glycan #5). All these results illustrate that the developed mucin-type O-glycosylation model framework can effectively simulate glycosylation of the skin tissue of the Atlantic Salmon, and identify the enzymes that are associated with the differentially expressed glycans. Our research therefore has suggested, albeit tentatively, three potentially important influences of tissue engineering, therapeutic target, and feeding strategy on the use of O-glycosylation models in the salmon industry. Owing to the generality of our O-glycosylation modeling framework, our O-glycosylation model could be applied in any species such as plant or animal (e.g., mammalian).

TABLE 1
Reaction rules for the mucin-type O-glycosylation model

| | | | Parsing |
|---|---|---|---|
| Gene Name+ | Substrate* | Product (Added Moiety) | Logic** |
| Galnt | ; Ser/Thr | (AN); Ser/Thr | Explicit |
| A3galnt | (AN); Ser/Thr | (ANa3AN); Ser/Thr | Explicit |
| B4galnt2 | (NNa3\hat{ }Ab | (ANb4) | Partial |
| ABO(A3galnt1) | (Fa2\hat{ }Ab | (ANa3) | Partial |
| B4galnt3/4_1 | (\hat{ }GNb3 | ANb4 | Partial |
| B4galnt3/4_2 | (\hat{ }GNb6 | ANb4 | Partial |
| pseudo_B4galnt2_1 | (\hat{ }Ab3)?AN | ANb4 | Partial |
| pseudo_B4galnt2_2 | (\hat{ }Ab{34}GNb6 | ANb4 | Partial |
| pseudo_B4galnt2_3 | (\hat{ }Ab{34}GNb3 | ANb4 | Partial |
| B4galt1/2/4/5_1 | (\hat{ }GNb3 | Ab4 | Partial |
| B4galt1/2/4/5_2 | (\hat{ }GNb6 | Ab4 | Partial |
| B3galt1/2_1 | (\hat{ }GNb3 | Ab3 | Partial |
| B3galt1/2_2 | (\hat{ }GNb6 | Ab3 | Partial |
| C1galt1 | (AN); Ser/Thr | (Ab3AN); Ser/Thr | Explicit |
| pseudo_C1gait1_1 | (ANa3AN); Ser/Thr; | (Ab3ANa3AN); Ser/Thr | Explicit |
| pseudo_C1gait1_2 | (NNa6(ANa3)AN); Ser/ | (NNa6(Ab3ANa3)AN); Ser/Thr | Explicit |
| | Thr | | |
| ABO(A3galt1) | (Fa2\hat{ }Ab4 | (Aa3) | Partial |
| B3gnt3_1 | (GNb6(Ab3)AN); Ser/ | (GNb6(GNb3Ab3)AN); Ser/Thr | Explicit |
| | Thr | | |
| B3gnt6 | (AN); Ser/Thr | (GNb3AN); Ser/Thr | Explicit |
| Gcnt1 | (Ab3AN); Ser/Thr | (GNb6(Ab3)AN); Ser/Thr | Explicit |
| pseudo_Gcnt1 | (Ab3ANa3AN); Ser/Thr | (GNb6(Ab3ANa3)AN); Ser/Thr | Explicit |
| pseudo_B3gnt3_1 | (GNb6(Ab3)ANa3AN); | (GNb6(GNb3Ab3)ANa3AN); Ser/Thr | Explicit |
| | Ser/Thr | | |
| pseudo_B3gnt3_2 | (NNa6(Ab3ANa3)AN); | (NNa6(GNb3Ab3ANa3)AN); Ser/Thr | Explicit |
| | Ser/Thr | | |
| pseudo_B3gnt3_3 | (*Ab3ANa3AN | GNb3 | Partial |
| pseudo_B3gnt3_4 | (*Ab3AN | GNb3 | Partial |
| Gcnt3 | (GNb3AN); Ser/Thr | (GNb3(GNb6)AN); Ser/Thr | Explicit |
| B3gnt2/4/5 | (\hat{ }Ab4GN | GNb3 | Partial |
| Gcnt2 | (Ab4GNb3\hat{ }Ab4 | (GNb6) | Partial |
| Fut3/5 | (Ab3GNb | (Fa4(Ab3)GNb | Replacement |
| Fut2 | (Ab3GNb | (Fa2Ab3GNb | Replacement |
| Fut1 | (Ab4GNb | (Fa2Ab4GNb | Replacement |
| Fut4/6/9 | (Ab4GNb | (Fa3(Ab4)GNb | Replacement |
| pseudo_Fut4/6/9 | (ANb4GNb | (Fa3(ANb4)GNb | Replacement |
| Fut7 | (NNa3Ab4GNb | (Fa3(NNa3Ab4)GNb | Replacement |
| pseudo_ANFut_1 | (ANb4Ab | (Fa3ANb4Ab | Replacement |
| pseudo_ANFut_2 | (ANb4GN | (Fa3ANb4GN | Replacement |
| pseudo_GNFut_1 | (GNb3Ab | (Fa3GNb3Ab | Replacement |
| pseudo_GNFut_2 | (GNb3AN | (Fa3GNb3AN | Replacement |
| pseudo_core5Fut_1 | (ANa3AN | (Fa3ANa3AN | Replacement |
| pseudo_core5Fut_2 | (NNa6(ANa3)AN | (NNa6(Fa3ANa3)AN | Replacement |
| St6gal1 | (\hat{ }Ab4GN | NNa6 | Partial |
| St6galnac1/2_1 | (Ab3AN); Ser/Thr | (NNa6(Ab3)AN); Ser/Thr | Explicit |
| St6galnac1/2_2 | (AN); Ser/Thr | (NNa6AN); Ser/Thr | Explicit |
| St6galnac3/4 | (NNa3Ab3AN); Ser/Thr | (NNa6(NNa3Ab3)AN); Ser/Thr | Explicit |
| St3gal1/2 | (?\hat{ }Ab3)?AN | NNa3 | Partial |
| St3gal3/4/6′ | (Ab4GN | NNa3 | Partial |
| pseudo_St6galnac1/2 | (ANa3AN); Ser/Thr | (NNa6ANa3AN); Ser/Thr | Explicit |
| pseudo_NGt6galnac1 | (ANa3AN); Ser/Thr | (NGa6(ANa3)AN); Ser/Thr | Explicit |
| pseudo_NGt6galnac12 | (Ab3AN); Ser/Thr | (NGa6(Ab3)AN); Ser/Thr | Explicit |
| pseudo_NGt6galnac3 | (AN); Ser/Thr | (NGa6AN); Ser/Thr | Explicit |
| Chst1 | Ab4GNb | [6S] | Partial |
| Chst2/4/5/6 | GN\hat{ }b6(Ab3)AN | [6S] | Partial |
| Chst2 | GN\hat{ }b3Ab4GN | [6S] | Partial |
| Chst4 | GN\hat{ }b3AN | [6S] | Partial |
| Gal3st2 | A\hat{ }b3GN | [3S] | Partial |
| Gal3st2/3 | A\hat{ }b4)?GN | [3S] | Partial |
| Gal3st4 | Ab3)?AN | [3S] | Partial |

+Pseudo-genes are genes that can either catalyze biochemically similar reaction(s) or are suspected for the corresponding reactions based on literature.
*A caret symbol indicates the position of added moiety (Product/Added Moiety) in the recognized portion of substrate string (Recognized (Partial) Substrate); the question mark following a bracket (“?”) indicates that the substrate string with or without the bracket will be recognized; the numbers inside the curly brackets indicate the possible carbon numbers on the monosaccharide bonded on the right side of the right curly bracket.
**Explicit: The product string is fully defined; Partial: the product string is a combination of the full substrate string and the added moiety (Product/Added Moiety) placed at the position of the caret symbol in Recognized (Partial) Substrate string; Replacement: the product string is the full substrate string with the Recognized (Partial) Substrate string replaced by Product/Added Moiety.

TABLE 2
Experimental measured glycomics data of skin tissue
of salmon for the O-glycosylation Markov modelling

| m/z | Glycan structure | S1 | S2 | S3 | S4 | S5 |
|---|---|---|---|---|---|---|
| 384 | Galb1-3GalNAcol | 2.01 | 1.86 | 5.67 | 1.46 | 3.00 |
| 425 | GlcNAcb1-3GalNAcol | 0.44 | 0.80 | 1.61 | 0.31 | 2.51 |
| 425 | GalNAca1-3GalNAcol | 2.91 | 5.81 | 9.08 | 4.39 | 6.70 |
| 513 | NeuAca2-6GalNAcol | 60.88 | 50.60 | 37.09 | 45.07 | 32.64 |
| 529 | NeuGca2-6GalNAcol | 9.79 | 4.67 | 8.14 | 22.34 | 27.33 |
| 571 | Fuc-GalNAca1-3GalNAcol | 2.31 | 9.04 | 8.34 | 7.37 | 0.16 |
| 571 | Fuc-GlcNAcb1-3GalNAcol | 0.38 | 0.77 | 1.04 | 0.57 | 3.15 |
| 587 | Gal-GalNAca1-3GllcNAcol | 1.52 | 0.23 | 0.00 | 0.00 | 0.00 |
| 587 | Galb1-3(GlcNAcb1-6)GalNAcol | 1.24 | 0.16 | 2.47 | 1.18 | 4.39 |
| 587 | Galb1-4GlcAcb1-3GalNAcol | 0.49 | 0.32 | 0.50 | 0.50 | 0.50 |
| 675 | Galb1-3(NeuAca2-6)GalNAcol | 1.06 | 0.78 | 1.35 | 0.78 | 0.00 |
| 675 | NeuAca2-3Galb1-3GalNAcol | 0.46 | 0.11 | 0.15 | 0.17 | 0.00 |
| 691 | Galb1-3(NeuGca2-6)GalNAcol | 0.12 | 0.12 | 0.24 | 0.25 | 0.00 |
| 716 | GlcNAcb1-3(NeuAca2-6)GalNAcol | 0.35 | 0.42 | 0.41 | 0.24 | 0.38 |
| 716 | GalNAca1-3(NeuAca2-6)GalNAcol | 3.90 | 11.89 | 12.99 | 4.70 | 5.34 |
| 732 | GalNAca1-3(NeuGca2-6)GalNAcol | 1.19 | 2.19 | 3.84 | 2.77 | 3.11 |
| 733 | Fuc-HexNAc-Galb1-3GalNAcol | 0.00 | 0.00 | 0.00 | 0.00 | 0.00 |
| 733 | Fuc-HexNAc-Galb1-3GalNAcol | 0.00 | 0.00 | 0.00 | 0.35 | 0.00 |
| 749 | Galb1-3(Galb1-4GlcNAcb1-6)GalNAcol | 0.70 | 0.28 | 1.20 | 0.00 | 0.00 |
| 749 | Galb1-4GlcNAcb1-3Galb1-3GalNAcol | 0.27 | 0.21 | 0.44 | 0.00 | 0.00 |
| 790 | HexNAc-Gal-GalNAca1-3GalNAcol | 0.30 | 0.00 | 0.00 | 0.00 | 0.00 |
| 790 | HexNAc-Galb1-3(GlcNAcb1-6)GalNAcol | 0.00 | 0.00 | 0.00 | 0.00 | 0.00 |
| 790 | HexNAc-(Gal)GalNAca1-3GalNAcol | 0.88 | 0.00 | 0.00 | 0.00 | 1.88 |
| 790 | Galb1-3(HexNAc-GlcNAcb1-6)GalNAcol | 0.00 | 0.63 | 0.76 | 0.54 | 0.00 |
| 862 | Fuc-GalNAca1-3(NeuAca2-6)GalNAcol | 2.09 | 2.74 | 2.02 | 1.41 | 0.85 |
| 878 | Fuc-GalNAca1-3(NeuGca2-6)GalNAcol | 0.50 | 0.49 | 0.54 | 0.58 | 0.45 |
| 878 | NeuAca2-3HexNAc1-4Galb1-3GalNAcol | 0.00 | 0.00 | 0.00 | 0.00 | 0.00 |
| 878 | Hex-GlcNAcb1-3(NeuAca2-6)GalNAcol | 0.40 | 0.69 | 0.26 | 0.23 | 0.11 |
| 878 | HexNAc-Galb1-3(NeuAca2-6)GalNAcol | 0.00 | 0.00 | 0.00 | 0.00 | 0.00 |
| 878 | Hex-GlcNAca1-3(NeuAca2-6)GalNAcol | 2.01 | 0.25 | 0.15 | 0.00 | 2.09 |
| 878 | NeuAca2-3Galb1-3(GlcNAcb1-6)GalNAcol | 0.00 | 0.00 | 0.00 | 0.00 | 0.00 |
| 894 | Hex-GalNAca1-3(NeuGca2-6)GalNAcol | 0.34 | 0.00 | 0.00 | 0.08 | 0.75 |
| 895 | Galb1-3[Gal(Fuc)GlcNAcb1-6]GalNAcol | 0.00 | 0.00 | 0.00 | 0.00 | 0.00 |
| 936 | HexNAc(Fuc)HexNAc-Galb1-3GalNAcol | 0.39 | 1.86 | 1.16 | 2.06 | 0.36 |
| 936 | Fuc-HexNAc-Gal-GalNAca1-3GalNAcol | 0.29 | 0.00 | 0.00 | 0.00 | 1.46 |
| 936 | Fuc-HexNAc-Galb1-3(GlcNAcb1-6)GalNAcol | 0.00 | 0.00 | 0.00 | 0.00 | 0.00 |
| 952 | HexNAc-Galb1-3(Galb1-4GlcNAcb1-6)GalNAcol | 0.00 | 0.00 | 0.00 | 0.00 | 0.00 |
| 952 | Gal-GlcNAc(Gal)GalNAca1-3GalNAcol | 0.16 | 0.00 | 0.00 | 0.00 | 0.00 |
| 966 | NeuAca2-3Galb1-3(NeuAca2-6)GalNAcol | 0.94 | 0.00 | 0.00 | 0.00 | 0.00 |
| 1227 | GalNAcb1-4(Fuca1-3)GlcNAc-Galb1-3(NeuAca2- | 0.00 | 1.49 | 0.00 | 1.12 | 0.00 |
| | 6)GalNAcol | | | | | |

### Example 3

### Lipidomics Markov Model Effectively Predicts a Comprehensive Lipidome of Human Plasma Sample

Lipids are essential for a variety of biological functions and are some of the most fundamental components of cells. While advances in lipidomics technology have enabled us to probe the pathogenesis of many severe but common diseases, such as hepatic steatosis, systematic study of shift in lipidomic mechanisms remains a daunting task due to the tremendous number of lipid subspecies and unclarified enzymes of analogous reactions (Han et al, 2016; Lydic et al, 2018). Previously published kinetic lipidomics models (ODEs) or FBA models only attempted to look at limited pools of specific, well characterized lipid species but often omitted disambiguating isomers and required a priori estimation of kinetic/constraint parameters, while higher-level, community-based lipid network analyses provided only limited insights into the genetic bases of lipidomic changes (Shih et al, 2008; McAuley & Mooney et al, 2015; Schützhold et al, 2016; Tsouka & Hatzimanikatis et al, 2020). Here, we extended our low-parameter Markov framework (Spahn et al., 2016; Liang et al., 2020) for modeling the complex synthesis process of the lipidome. Fully realizing the potential of Markov modeling, it is of great interest for us to understand and quantify the underlying lipid synthesis dynamics when presented with comprehensive lipidomics data.

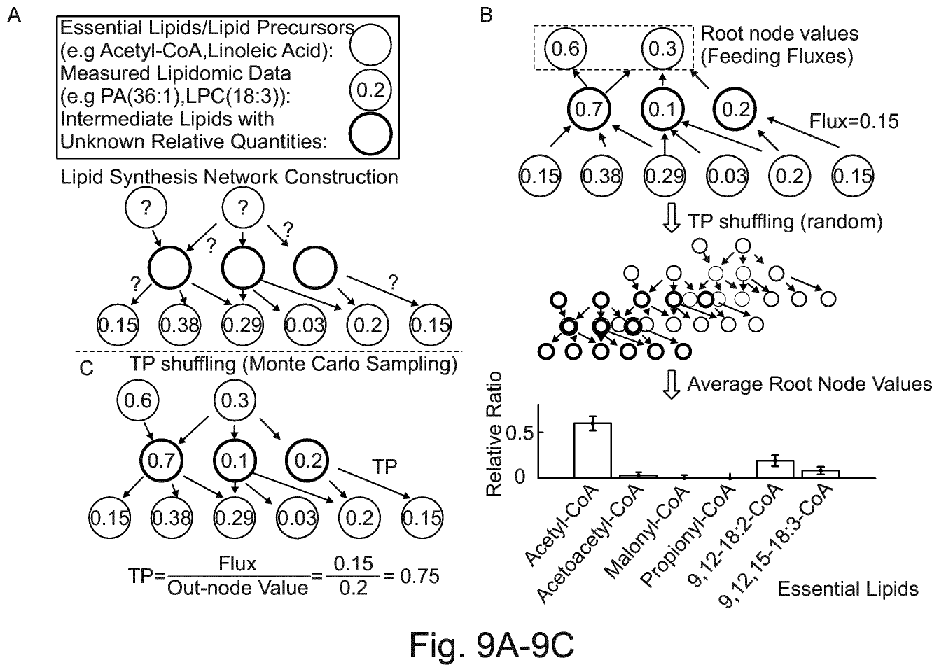

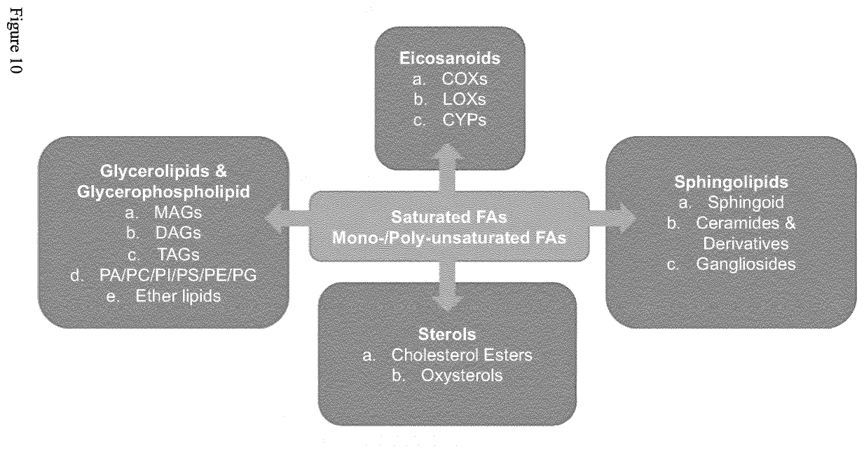

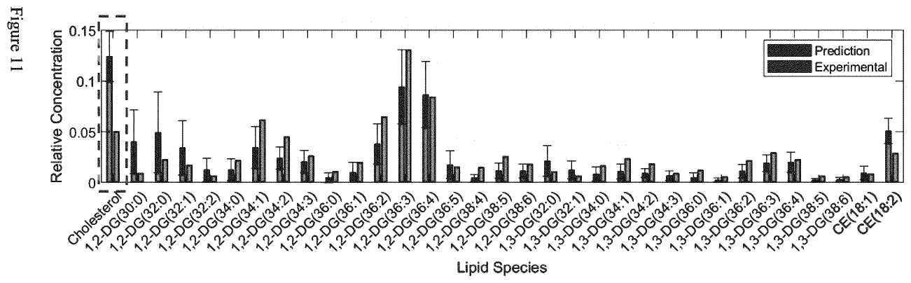

A collaboration between NIST and NIDDK (Quehenberger et al, 2010) published a large set of comprehensive lipidomic samples (100 human plasma lipidomic samples) with 500 measured lipid subspecies. In this study, we developed a comprehensive lipidomic model which predicted a lipidomics sample of the human plasma dataset. Table 3 shows the reaction rules for constructing the lipid synthesis network used to demonstrate the modeling framework, and the scope of the modeling framework was summarized in FIG. 10. Due to the highly complex lipid synthesis network and a large number of reaction rules (variables), the original stochastic global optimization method became less feasible as the fitting time increased exponentially. To overcome this challenge, we therefore employed an alternative method (FIG. 9) to approximate the transition probabilities for the lipidomic Markov models. Briefly, after the construction of the lipid synthesis network (FIG. 9A), all the edges in the network were reversed, allowing the measured lipid values to randomly walk through the network via randomly shuffled transition probabilities (FIG. 9B). Multiple models were generated and the solution spaces of feeding fluxes (relative concentrations of essential fatty acids) as well as all intermediate fluxes were sampled. The edges in the network were then reversed again and a set of transition probabilities was calculated for each sample via Monte Carlo sampling of the fluxes generated from the previous step (FIG. 9C), thus providing us the fitted parameters. In essence, the method is based on the assumption that a given set of lipidomics data is comprehensive enough to strictly define the solution spaces of the feeding fluxes (the relative concentrations of essential lipids), and to a certain extent, portraying the solution spaces of other intermediate lipids. Indeed, we observed that the sampled Markov models via random walk produced the feeding fluxes of the essential lipids with relatively small variances (e.g. the standard deviation of fitted relative concentrations for acetyl-CoA, a major FA synthesis precursors, was only 12.5%, FIG. 9C). FIG. 11 shows the fitted lipidomic profile vs. the experimentally measured profile (top 129 signals). The resulting models showed great performance in predicting the experimentally measured lipidomics data (RMSE=9.9e-3). While there was discrepancy between the measured and the fitted cholesterol values, it could be explained by the fact that dietary cholesterol is usually abundant and contributes significantly to the plasma level beyond de novo synthesis. These results have demonstrated that our lipid Markov model could effectively model the complex lipid synthesis, and our method could shed light on understanding the pathogenesis of many lipid-related disorders.

TABLE 3
Reaction rules (partial) for the lipidomics Markov model^{#}

| | Gene/ | | | |
|---|---|---|---|---|
| Synthesis Type | Enzyme* | Reactant(s) | Product | Constraints |
| Saturated FA | FASN_1 | Malonyl-CoA; Acetyl-CoA | 4: 0-CoA | |
| Saturated FA | FASN_2 | Malonyl-CoA; Propionyl-CoA | 5: 0-CoA | |
| Saturated FA | FASN_k | n: 0-CoA; Acetyl-CoA | (n + 2): 0-CoA | 4 <= n <= 17 |
| Mono-unsaturated FA | MUFADS1_k | n: 0-CoA | (n − 7) − n: 1-CoA | 14 <= n <= 19 |
| Mono-unsaturated FA | MUFADS2_k | n: 0-CoA | (n − 9) − n: 1-CoA | 14 <= n <= 19 |
| Multi-unsaturated | FADS_1 | 9, 12-18: 2-CoA | 6, 9, 12-18: 3-CoA | |
| Multi-unsaturated | FADS_2 | 8, 11, 14-20: 3-CoA | 5, 8, 11, 14-20: 4-CoA | |
| Multi-unsaturated | FADS_3 | 7, 10, 13, 16-22: 4-CoA | 4, 7, 10, 13, 16-22: 5- | |
| | | | CoA | |
| Multi-unsaturated | FADS_4 | 9, 12, 15, 18-24: 4-CoA | 6, 9, 12, 15, 18-24: 5- | |
| | | | CoA | |
| Multi-unsaturated | FADS_5 | 9, 12, 15-18: 3-CoA | 6, 9, 12, 15-18: 4-CoA | |
| Multi-unsaturated | FADS_6 | 8, 11, 14, 17-20: 4-CoA | 5, 8, 11, 14, 17-20: 5- | |
| | | | CoA | |
| Multi-unsaturated | FADS_7 | 7, 10, 13, 16, 19-22: 5-CoA | 4, 7, 10, 13, 16, 19-22: 6- | |
| | | | CoA | |
| Multi-unsaturated | FADS_8 | 7, 10, 13, 16-22: 4-CoA | 9, 12, 15, 18-24: 4-CoA | |
| Multi-unsaturated | FADS_9 | 9, 12, 15, 18, 21-24: 5-CoA | 6, 9, 12, 15, 18, 21- | |
| | | | 24: 6-CoA | |
| VLCFA | ELOVL_1 | 6, 9, 12-18: 3-CoA; Acetyl-CoA | 8, 11, 14-20: 3-CoA | |
| VLCFA | ELOVL_2 | 5, 8, 11, 14-20: 4-CoA; Acetyl-CoA | 7, 10, 13, 16-22: 4-CoA | |
| VLCFA | ELOVL_3 | 6, 9, 12, 15-18: 4-CoA; Acetyl-CoA | 8, 11, 14, 17-20: 4-CoA | |
| VLCFA | ELOVL_4 | 5, 8, 11, 14, 17-20: 5-CoA; Acetyl-CoA | 7, 10, 13, 16, 19-22: 5- | |
| | | | CoA | |
| VLCFA | ELOVL_5 | 7, 10, 13, 16, 19-22: 5-CoA; Acetyl-CoA | 9, 12, 15, 18, 21-24: 5- | |
| | | | CoA | |
| Eicosanoids | COX_1 | 8, 11, 14-20: 3-CoA | PG1 | |
| Eicosanoids | COX_2 | 5, 8, 11, 14-20: 4-CoA | PG2 | |
| Eicosanoids | COX_3 | 5, 8, 11, 14, 17-20: 5-CoA | PG3 | |
| PA | GPAT_k | . . . −n: x-CoA; G3P | LPA(n1: x1) | 14 <= n <= 22, |
| | | | | x <= 6 |
| PA | AGPAT_k | LPA(n1: x1); . . . −n2: x2-CoA | PA(n1 + n2: x1 + x2) | 14 <= n <= 22, |
| | | | | x1 + x2 <= 9, |
| | | | | abs(n1 − n2) = |
| | | | | 0 or 2 |
| Diacylglycerol/ | PAP_k | PA(n1 + n2: x1 + x2) | 1_2- | |
| Triacylglycerol | | | DG(n1 + n2: x1 + x2) | |
| Diacylglycerol/ | DAG_isomerization_k | 1_2-DG(n1 + n2: x1 + x2) | 1_3- | |
| Triacylglycerol | | | DG(n1 + n2: x1 + x2) | |
| Diacylglycerol/ | DGAT_k | 1_2-DG(n1 + n2: x1 + x2) | TG(n1 + n2 + n3: x1 + | 14 <= n <= 22, |
| Triacylglycerol | | | x2 + x3) | x1 + x2 + x3 <= |
| | | | | 6, abs(n1 − |
| | | | | n3) = 0 or 2, |
| | | | | abs(n2 − |
| | | | | n3) = 0 or 2 |
| Sterol | HSD3B | Cholesterol | Cholestenone | |
| Sterol | HMGCS; | Acetyl-CoA; Acetoacetyl-CoA | 3,3- | |
| | HMGCR; MVK; | | dimethylallylpyrophosphoric acid | |
| | PMVK; MVD | | | |
| Sterol | IDI1 | 3,3-dimethylallylpyrophosphoric acid | 5- | |
| | | | isopentenylpyrophosphoric acid | |
| Sterol | FDPS; GGPS; | 3,3-dimethylallylpyrophosphoric | Lanosterol | |
| | FDFT; SQLE; | acid; 5-isopentenylpyrophosphoric acid | | |
| | LSS | | | |
| Sterol | Cyp51_1; | Lanosterol | 24-dehydrolathosterol | |
| | LBR_1; | | | |
| | NSDHL_1 | | | |
| Sterol | SC5D_1 | 24-dehydrolathosterol | 7- | |
| | | | dehydrodesmosterol | |
| Sterol | DHCR7_1 | 7-dehydrodesmosterol | Desmosterol | |
| Sterol | DHCR24_1 | Lanosterol | Cholesta-8(9)-en-3B- | |
| | | | ol | |
| Sterol | Cyp51_2; | Cholesta-8(9)-en-3B-ol | Lathosterol | |
| | LBR_2; | | | |
| | NSDHL_2 | | | |
| Sterol | SC5D_2 | Lathosterol | 7-dehydrocholesterol | |
| Sterol | DHCR7_2 | 7-dehydrocholesterol | Cholesterol | |
| Sterol | DHCR24_2 | Desmosterol | Cholesterol | |
| Sterol | ACAT | Cholesterol; . . . −n: x-CoA | CE(n1: x1) | 14 <= n <= 22, |
| | | | | x <= 4 |
| Sterol | Cyp7A1 | Cholesterol | 7alpha-hydroxy- | |
| | | | cholesterol | |
| Sterol | Cyp3A4 | Cholesterol | 4beta-hydroxy- | |
| | | | cholesterol | |
| Sterol | Cyp46A1 | Cholesterol | 24S-hydroxy- | |
| | | | cholesterol | |
| Sterol | CH25H | Cholesterol | 25-hydroxy- | |
| | | | cholesterol | |
| Sterol | CYP27A1_1 | Cholesterol | 27-hydroxy- | |
| | | | cholesterol | |
| Sterol | ROS_1 | Cholesterol | 7-oxo-cholesterol | |
| Sterol | LCAT | 7-oxo-cholesterol | 7-oxo-cholesterol | |
| | | | ester | |
| Sterol | SOAT1/2 | 7-oxo-cholesterol | 7-oxo-cholesterol | |
| | | | ester | |
| Sterol | Cyp27A1_2 | 7-oxo-cholesterol | 27-hydroxy-7-oxo- | |
| | | | cholesterol | |
| Sterol | HSD11B1 | 7-oxo-cholesterol | 7beta-hydroxy- | |
| | | | cholesterol | |
| PC | CPT; PLC_k | CDP-choline; | PC(n1 + n2: x1 + x2) | 16 <= n <= 22 |
| | | 1_2-DAG(n1 + n2: x1 + x2) | | |
| PC | PEMT_k | PE(n1 + n2: x1 + x2) | PC(n1 + n2: x1 + x2) | 16 <= n <= 22 |
| PC | PLA2G6_1; | PC(n1 + n2: x1 + x2) | LPC(n1: x1); . . . n2: | 16 <= n <= 22 |
| | LPCAT_1_k | | x2-CoA | |
| PA | PLD_k | PC(n1 + n2: x1 + x2) | PA(n1 + n2: x1 + x2) | 16 <= n <= 22 |
| PE | EPT_k | CDP-ethanolamine; 1_2- | PE(n1 + n2: x1 + x2) | 16 <= n <= 22 |
| | | DG(n1 + n2: x1 + x2) | | |
| PE | PLA2G6_2; | PE(n1 + n2: x1 + x2) | LPE(n1: x1) | 16 <= n <= 22 |
| | LPCAT_2_k | | | |
| PG | CDS1; PGS; | PA(n1 + n2: x1 + x2); G3P | PG(n1 + n2: x1 + x2) | 16 <= n <= 22 |
| | PTPMT_k | | | |
| PI | CDIPT_k | PA(n1 + n2: x1 + x2) | PI(n1 + n2: x1 + x2) | 16 <= n <= 22 |
| PS | PTDSS1_k | PC(n1 + n2: x1 + x2) | PS(n1 + n2: x1 + x2) | 16 <= n <= 22 |
| PS | PTDSS2_k | PE(n1 + n2: x1 + x2) | PS(n1 + n2: x1 + x2) | 16 <= n <= 22 |
| Ether Lipid | GNPAT; | G3P; . . . n: x-CoA | 1-O-alkyl(n: x) | n = 16, 18, 20, |
| | AGPS; | | | 22 |
| | R03455(etc)_k | | | |
| Ether Lipid | EPT_like_k | . . . n2: x2-CoA; 1-O-alkyl(n1: x1) | PE(n1: x1) | x <= 7, |
| | | | | abs(n1 − |
| | | | | n2) <= 2 |
| Ether Lipid | CPT_like_k | . . . n2: x2-CoA; 1-O-alkyl(n1: x1) | PC(n1x1) | x <= 7, |
| | | | | abs(n1 − |
| | | | | n2) <= 2 |
| Ether Lipid | PEDS1_1_k | PE(n1 + n2: x1 + x2e) | PE(n1: x1) | |
| Ether Lipid | PEDS1_2_k | PC(n1 + n2: x1 + x2e) | PC(n1: x1) | |
| Ether Lipid | PLA2G6_3; | PE(n1 + n2: x1 + x2e) | LPE(n1: x1e) | |
| | LPCAT_3_{—} | | | |
| Ether Lipid | PLA2G6_4; | PC(n1 + n2: x1 + x2e) | LPC(n1: x1e) | |
| | LPCAT_4_{—} | | | |
| Ether Lipid | PLA2G6_5; | PE(n1 + n2: x1 + x2p) | LPE(n1: x1p) | |
| | LPCAT_5_{—} | | | |
| Ether Lipid | PLA2G6_6; | PC(n1 + n2: x1 + x2p) | LPC(n1: x1p) | |
| | LPCAT_6_{—} | | | |

^{#}Potential retro-conversions and short-path inter-conversions between lipid species are implicitly combined with the corresponding synthesis reactions, or ignored as they usually happen in mitochondria and peroxisome.
*Genes/Enzymes ending with _k means the reactions are dynamically numbered, based on the number of possible lipid species as their corresponding substrates.

TABLE 4
Experimentally measured lipidomics data of a human plasma sample*

| | Relative | | Relative | | Relative |
|---|---|---|---|---|---|
| Lipid Species | Concentration | Lipid Species | Concentration | Lipid Species | Concentration |
| 10Z-heptadecenoic acid | 16.51 | PE(38:6) | 311.19 | 1_2-DG(32:3) | 949.86 |
| 11_14_17-eicosatrienoic | 5.45 | PE(40:1) | 27.60 | 1_2-DG(34:0) | 21,871.37 |
| acid | | | | | |
| 11_14-eicosadienoic acid | 5.62 | PE(40:4) | 37.43 | 1_2-DG(34:1) | 61,836.99 |
| 5_8_11_14_17- | 6.95 | PE(40:5) | 77.29 | 1_2-DG(34:2) | 45,032.09 |
| eicosapentaenoic acid | | | | | |
| 5_8_11-eicosatrienoic | 1.53 | PE(40:6) | 159.94 | 1_2-DG(34:3) | 26,354.09 |
| acid | | | | | |
| 7_10_13_16_19- | 6.39 | PE(40:7) | 41.02 | 1_2-DG(34:4) | 2,756.87 |
| docosapentaenoic acid | | | | | |
| 7Z_10Z_13Z_16Z- | 5.82 | PE(42:1) | 13.78 | 1_2-DG(36:0) | 11,020.12 |
| docosatetraenoic acid | | | | | |
| 9Z-palmitoleic acid | 234.07 | PE(42:5) | 23.47 | 1_2-DG(36:1) | 19,855.78 |
| alpha-linolenic acid | 1.83 | PE(42:6) | 26.16 | 1_2-DG(36:2) | 64,902.29 |
| Arachidic acid | 3.80 | PE(42:7) | 19.09 | 1_2-DG(36:3) | 130,720.79 |
| Arachidonic acid | 46.94 | PG(34:1) | 9.66 | 1_2-DG(36:4) | 84,294.14 |
| Behenic acid | 2.55 | PG(34:2) | 1.27 | 1_2-DG(36:5) | 15,358.31 |
| bishomo-gamma- | 8.66 | PG(36:1) | 25.34 | 1_2-DG(38:0) | 52.88 |
| linolenic acid | | | | | |
| Cerotic acid | 1.77 | PG(36:2) | 3.09 | 1_2-DG(38:1) | 210.83 |
| cis-selacholeic acid | 1.12 | PG(36:3) | 5.40 | 1_2-DG(38:2) | 355.78 |
| DHA | 15.82 | PG(36:4) | 3.47 | 1_2-DG(38:3) | 4,523.35 |
| gamma-linolenic acid | 16.50 | PG(36:5) | 5.60 | 1_2-DG(38:4) | 14,981.81 |
| Lauric acid | 11.49 | PG(38:4) | 3.06 | 1_2-DG(38:5) | 25,764.75 |
| Lignoceric acid | 4.19 | PG(38:5) | 9.67 | 1_2-DG(38:6) | 18,171.04 |
| Linoleic acid | 243.42 | PG(38:6) | 14.68 | 1_2-DG(40:4) | 576.40 |
| Margaric acid | 19.10 | PG(40:4) | 5.16 | 1_2-DG(40:6) | 4,057.98 |
| Myristic acid | 96.85 | PG(40:5) | 2.54 | 1_2-DG(40:7) | 4,956.34 |
| Oleic acid | 1,283.55 | PG(40:6) | 2.32 | 1_3-DG(30:0) | 3,886.93 |
| Palmitic acid | 1,018.88 | PG(40:7) | 2.90 | 1_3-DG(30:1) | 1,679.47 |
| Pentadecanoic acid | 10.43 | PG(40:8) | 1.68 | 1_3-DG(30:2) | 210.45 |
| Stearic acid | 352.87 | PG(40:9) | 1.85 | 1_3-DG(32:0) | 10,816.64 |
| LPC(16:0) | 476.17 | PI(32:1) | 11.91 | 1_3-DG(32:1) | 6,561.42 |
| LPC(16:1) | 60.23 | PI(34:0) | 7.55 | 1_3-DG(32:2) | 2,564.41 |
| LPC(18:0) | 372.68 | PI(34:1) | 28.36 | 1_3-DG(32:3) | 274.41 |
| LPC(18:1) | 236.68 | PI(34:2) | 42.15 | 1_3-DG(34:0) | 16,653.51 |
| LPC(18:2) | 269.38 | PI(36:0) | 4.30 | 1_3-DG(34:1) | 23,664.91 |
| LPC(20:3) | 52.15 | PI(36:1) | 28.34 | 1_3-DG(34:2) | 18,591.36 |
| LPC(20:4) | 91.57 | PI(36:2) | 68.51 | 1_3-DG(34:3) | 9,118.27 |
| LPC(22:5) | 16.00 | PI(36:3) | 18.62 | 1_3-DG(34:4) | 732.22 |
| LPC(22:6) | 25.14 | PI(36:4) | 20.22 | 1_3-DG(36:0) | 12,293.16 |
| LPE(16:0) | 55.17 | PI(36:5) | 9.10 | 1_3-DG(36:1) | 5,767.59 |
| LPE(18:0) | 118.35 | PI(38:2) | 3.02 | 1_3-DG(36:2) | 21,768.49 |
| LPE(18:1) | 102.92 | PI(38:3) | 29.22 | 1_3-DG(36:3) | 29,429.99 |
| LPE(18:2) | 125.39 | PI(38:4) | 175.82 | 1_3-DG(36:4) | 22,673.99 |
| LPE(20:4) | 116.89 | PI(38:5) | 20.39 | 1_3-DG(36:5) | 4,987.01 |
| LPE(22:1) | 4.97 | PI(38:6) | 7.96 | 1_3-DG(38:0) | 632.74 |
| LPE(22:6) | 61.62 | PI(40:3) | 3.47 | 1_3-DG(38:1) | 36.10 |
| PA(32:0) | 2.97 | PI(40:4) | 7.20 | 1_3-DG(38:2) | 113.67 |
| PA(32:1) | 3.17 | PI(40:5) | 7.59 | 1_3-DG(38:3) | 572.99 |
| PA(34:0) | 3.59 | PI(40:6) | 8.62 | 1_3-DG(38:4) | 4,037.42 |
| PA(34:1) | 2.36 | PS(32:1) | 3.82 | 1_3-DG(38:5) | 6,382.06 |
| PA(34:2) | 2.30 | PS(34:0) | 4.91 | 1_3-DG(38:6) | 5,751.82 |
| PA(36:0) | 3.69 | PS(34:1) | 2.97 | 1_3-DG(40:4) | 707.83 |
| PA(36:1) | 1.89 | PS(34:2) | 2.42 | 1_3-DG(40:6) | 1,885.93 |
| PA(36:2) | 3.57 | PS(36:0) | 33.34 | 1_3-DG(40:7) | 1,378.54 |
| PA(36:3) | 1.87 | PS(36:1) | 11.71 | CE(14:0) | 1,278.05 |
| PA(36:4) | 3.31 | PS(36:2) | 5.05 | CE(14:1) | 484.06 |
| PA(38:2) | 2.20 | PS(36:3) | 1.97 | CE(15:0) | 468.09 |
| PA(38:3) | 1.93 | PS(36:4) | 2.86 | CE(15:1) | 468.09 |
| PA(38:4) | 2.63 | PS(38:1) | 4.21 | CE(16:0) | 3,046.56 |
| PA(38:5) | 3.38 | PS(38:2) | 3.00 | CE(16:1) | 1,789.27 |
| PA(38:6) | 1.11 | PS(38:3) | 3.10 | CE(16:2) | 500.04 |
| PC(30:1) | 17.82 | PS(38:4) | 7.68 | CE(17:0) | 538.38 |
| PC(32:0) | 182.87 | PS(38:5) | 4.49 | CE(17:1) | 538.38 |
| PC(32:1) | 457.56 | PS(38:6) | 2.58 | CE(18:0) | 915.41 |
| PC(32:2) | 156.56 | PS(40:3) | 1.44 | CE(18:1) | 8,499.05 |
| PC(34:0) | 122.38 | PS(40:4) | 1.62 | CE(18:2) | 29,016.59 |
| PC(34:1) | 1,426.20 | PS(40:5) | 1.82 | CE(18:3) | 2,332.45 |
| PC(34:2) | 2,996.77 | PS(40:6) | 10.17 | CE(20:0) | 511.22 |
| PC(34:3) | 220.68 | PS(40:7) | 2.70 | CE(20:1) | 495.25 |
| PC(36:0) | 126.99 | 24S-hydroxy- | 0.80 | CE(20:2) | 547.97 |
| | | cholesterol | | | |
| PC(36:1) | 1,594.33 | 25-hydroxy- | 0.27 | CE(20:3) | 527.20 |
| | | cholesterol | | | |
| PC(36:2) | 4,051.88 | 27-hydroxy- | 8.52 | CE(20:4) | 3,802.21 |
| | | cholesterol | | | |
| PC(36:3) | 2,628.01 | 4beta-hydroxy- | 0.85 | CE(22:0) | 346.67 |
| | | cholesterol | | | |
| PC(36:4) | 2,745.56 | 7 alpha-hydroxy- | 5.32 | CE(22:1) | 116.62 |
| | | cholesterol | | | |
| PC(36:5) | 204.37 | 7-oxo- | 1.97 | LPC(16:0e) | 7.84 |
| | | cholesterol | | | |
| PC(38:2) | 600.57 | Cholestenone | 1.23 | LPC(16:0p) | 27.56 |
| PC(38:4) | 4,061.40 | Cholesterol | 50,329.19 | LPC(18:0e) | 14.56 |
| PC(38:5) | 1,379.44 | Desmosterol | 28.86 | PC(34:1e) | 44.74 |
| PC(38:6) | 1,005.24 | Lanosterol | 7.72 | PC(34:2e) | 63.71 |
| PC(40:2) | 2,132.19 | Lathosterol | 85.90 | PC(36:1e)/PC(36:0p) | 41.08 |
| PC(40:4) | 587.06 | TG(48:1) | 430.81 | PC(36:2e)/PC(36:1p) | 99.92 |
| PC(40:5) | 1,064.01 | TG(48:2) | 322.71 | PC(36:4e)/PC(36:3p) | 463.82 |
| PC(40:6) | 1,268.62 | TG(50:0) | 185.32 | PC(38:3e)/PC(38:2p) | 184.56 |
| PC(40:7) | 369.30 | TG(50:1) | 1,015.52 | PC(38:5e)/PC(38:4p) | 797.05 |
| PC(40:8) | 431.77 | TG(50:2) | 1,275.39 | PE(34:1p) | 62.79 |
| PE(32:1) | 14.44 | TG(50:3) | 912.74 | PE(34:2p) | 116.97 |
| PE(34:0) | 12.79 | TG(50:4) | 302.47 | PE(36:2e)/PE(36:1p) | 87.22 |
| PE(34:1) | 95.06 | TG(52:1) | 472.35 | PE(36:3e)/PE(36:2p) | 218.76 |
| PE(34:2) | 191.53 | TG(52:2) | 2,228.07 | PE(36:4e)/PE(36:3p) | 187.56 |
| PE(36:0) | 225.15 | TG(52:3) | 3,432.10 | PE(36:5e)/PE(36:4p) | 313.28 |
| PE(36:1) | 131.19 | TG(52:4) | 1,451.66 | PE(38:5e)/PE(38:4p) | 871.59 |
| PE(36:2) | 471.59 | TG(52:5) | 511.22 | PE(38:6e)/PE(38:5p) | 522.36 |
| PE(36:3) | 254.91 | TG(54:2) | 343.48 | PE(40:5e) | 123.02 |
| PE(36:4) | 365.33 | TG(54:3) | 1103.39 | PE(40:6e) | 184.39 |
| PE(36:5) | 65.45 | TG(54:4) | 1093.8 | PE(40:7e) | 253.39 |
| PE(38:1) | 165.31 | TG(54:5) | 856.83 | PE(42:5p) | 29.28 |
| PE(38:2) | 12.19 | TG(54:6) | 583.64 | PE(42:6p) | 27.15 |
| PE(38:3) | 109.16 | TG(56:6) | 374.9 | | |
| PE(38:4) | 768.36 | 1_2-DG(30:0) | 9213.64 | | |
| PE(38:5) | 333.94 | 1_2-DG(30:1) | 3686.22 | | |
| | | 1_2-DG(30:2) | 510.74 | | |
| | | 1_2-DG(32:0) | 22624.94 | | |
| | | 1_2-DG(32:1) | 17080.86 | | |
| | | 1_2-DG(32:2) | 6631.07 | | |

*The data was normalized when used for modeling. The dataset was collaboratively produced by NIST and NIDDK as a human plasma standard reference material (SRM 1950) (Quehenberger et al, 2020).

### REFERENCES

[1] Pinho S S, Reis C A. Glycosylation in cancer: mechanisms and clinical implications. Nat Rev Cancer. 2015; 15:540-55. doi:10.1038/nrc3982.
[2] Varki A, Cummings R D, Esko J D, Freeze H H, Stanley P, Bertozzi C R, et al., editors. Essentials of Glycobiology. 2nd edition. Cold Spring Harbor (NY): Cold Spring Harbor Laboratory Press; 2009. http://www.ncbi.nlm.nih.gov/books/NBK1908/. Accessed 29 June 2019.
[3] Reily C, Stewart T J, Renfrow M B, Novak J. Glycosylation in health and disease. Nat Rev Nephrol. 2019; 1. doi:10.1038/s41581-019-0129-4.
[4] Moremen K W, Tiemeyer M, Nairn A V. Vertebrate protein glycosylation: diversity, synthesis and function. Nat Rev Mol Cell Biol. 2012; 13:448-62. doi:10.1038/nrm3383.
[5] Luo C, Chen S, Xu N, Wang C, Sai W bo, Zhao W, et al. Glycoengineering of pertuzumab and its impact on the pharmacokinetic/pharmacodynamic properties. Sci Rep. 2017; 7:46347. doi:10.1038/srep46347.
[6] Schwarz D S, Blower M D. The endoplasmic reticulum: structure, function and response to cellular signaling. Cell Mol Life Sci. 2016; 73:79-94. doi:10.1007/s00018-015-2052-6.
[7] Bankaitis V A, Garcia-Mata R, Mousley C J. Golgi Membrane Dynamics and Lipid

Metabolism. Curr Biol. 2012; 22:R414-24. doi:10.1016/j.cub.2012.03.004.
[8] Hossler P. Protein Glycosylation Control in Mammalian Cell Culture: Past Precedents and Contemporary Prospects. In: Hu W S, Zeng A-P, editors. Genomics and Systems Biology of Mammalian Cell Culture. Berlin, Heidelberg: Springer Berlin Heidelberg; 2012. p. 187-219. doi:10.1007/10_2011_113.
[9] Umaña P, Bailey JE. A mathematical model of N-linked glycoform biosynthesis. Biotechnol Bioeng. 1997; 55:890-908.
[10] Krambeck F J, Betenbaugh M J. A mathematical model of N-linked glycosylation. Biotechnol Bioeng. 2005; 92:711-28. doi:10.1002/bit.20645.
[11] Liu G, Marathe D D, Matta K L, Neelamegham S. Systems-level modeling of cellular glycosylation reaction networks: O-linked glycan formation on natural selectin ligands. Bioinformatics. 2008; 24:2740-7. doi:10.1093/bioinformatics/btn515.
[12] Liu G, Neelamegham S. A Computational Framework for the Automated Construction of Glycosylation Reaction Networks. PLOS ONE. 2014; 9:e100939. doi:10.1371/journal.pone.0100939.
[13] McDonald A G, Tipton K F, Davey G P. A Knowledge-Based System for Display and Prediction of O-Glycosylation Network Behaviour in Response to Enzyme Knockouts. PLOS Comput Biol. 2016; 12:e1004844. doi:10.1371/journal.pcbi.1004844.
[14] Kremkow B G, Lee K H. Glyco-Mapper: A Chinese hamster ovary (CHO) genome-specific glycosylation prediction tool. Metab Eng. 2018; 47:134-42. doi:10.1016/j.ymben.2018.03.002.
[15] Spahn P N, Hansen A H, Hansen H G, Arnsdorf J, Kildegaard H F, Lewis N E. A Markov chain model for N-linked protein glycosylation—towards a low-parameter tool for model-driven glycoengineering. Metab Eng. 2016; 33:52-66. doi:10.1016/j.ymben.2015.10.007.
[16] Spahn P N, Hansen A H, Kol S, Voldborg B G, Lewis N E. Predictive glycoengineering of biosimilars using a Markov chain glycosylation model. Biotechnol J. 2017; 12. doi:10.1002/biot.201600489.
[17] Bydlinski N, Maresch D, Schmieder V, Klanert G, Strasser R, Borth N. The contributions of individual galactosyltransferases to protein specific N-glycan processing in Chinese Hamster Ovary cells. J Biotechnol. 2018; 282:101-10. doi:10.1016/j.jbiotec.2018.07.015.
[18] Hassinen A, Khoder-Agha F, Khosrowabadi E, Mennerich D, Harrus D, Noel M, et al. A Golgi-associated redox switch regulates catalytic activation and cooperative functioning of ST6Gal-I with B4GalT-I. Redox Biol. 2019; 24:101182.
[19] Ujita M, Misra A K, McAuliffe J, Hindsgaul O, Fukuda M. Poly-N-acetyllactosamine Extension in N-Glycans and Core 2- and Core 4-branched O-Glycans Is Differentially Controlled by i-Extension Enzyme and Different Members of the β1,4-Galactosyltransferase Gene Family. J Biol Chem. 2000; 275:15868-75. doi: 10.1074/jbc.M001034200.
[20] Mkhikian H, Mortales C-L, Zhou R W, Khachikyan K, Wu G, Haslam S M, et al. Golgi self-correction generates bioequivalent glycans to preserve cellular homeostasis. eLife. 2016; 5. doi:10.7554/eLife.14814.
[21] El-Battari A, Prorok M, Angata K, Mathieu S, Zerfaoui M, Ong E, et al. Different glycosyltransferases are differentially processed for secretion, dimerization, and autoglycosylation. Glycobiology. 2003; 13:941-53.
[22] Rohfritsch P F, Joosten J A F, Krzewinski-Recchi M-A, Harduin-Lepers A, Laporte B, Juliant S, et al. Probing the substrate specificity of four different sialyltransferases using synthetic β-d-Galp-(1→4)-β-d-GlcpNAc-(1→2)-α-d-Manp-(1→O) (CH2)7CH3 analogues: General activating effect of replacing N-acetylglucosamine by N-propionylglucosamine. Biochim Biophys Acta BBA—Gen Subj. 2006; 1760:685-92. doi:10.1016/j.bbagen.2005.12.012.
[23] Mondal N, Buffone A, Stolfa G, Antonopoulos A, Lau J T Y, Haslam S M, et al. ST3Gal-4 is the primary sialyltransferase regulating the synthesis of E-, P-, and L-selectin ligands on human myeloid leukocytes. Blood. 2015; 125:687-96.
[24] Yang Z, Wang S, Halim A, Schulz M A, Frodin M, Rahman S H, et al. Engineered CHO cells for production of diverse, homogeneous glycoproteins. Nat Biotechnol. 2015; 33:842-4.
[25] Krambeck F J, Bennun S V, Andersen M R, Betenbaugh M J. Model-based analysis of N-glycosylation in Chinese hamster ovary cells. PLOS ONE. 2017; 12:e0175376. doi:10.1371/journal.pone.0175376.
[26] Chuang G-Y, Boyington J C, Joyce M G, Zhu J, Nabel G J, Kwong P D, et al. Computational prediction of N-linked glycosylation incorporating structural properties and patterns. Bioinformatics. 2012; 28:2249-55. doi:10.1093/bioinformatics/bts426.
[27] Ito H, Kameyama A, Sato T, Sukegawa M, Ishida H-K, Narimatsu H. Strategy for the fine characterization of glycosyltransferase specificity using isotopomer assembly. Nat Methods. 2007; 4:577-82. doi:10.1038/nmeth1050.
[28] Stanley P, Schachter H, Taniguchi N. N-Glycans. In: Varki A, Cummings R D, Esko J D, Freeze H H, Stanley P, Bertozzi C R, et al., editors. Essentials of Glycobiology. 2nd edition. Cold Spring Harbor (NY): Cold Spring Harbor Laboratory Press; 2009. http://www.ncbi.nlm.nih.gov/books/NBK1917/. Accessed 29 Jun. 2019.
[29] Čaval T, Tian W, Yang Z, Clausen H, Heck A J R. Direct quality control of glycoengineered erythropoietin variants. Nat Commun. 2018; 9. doi:10.1038/s41467-018-05536-3.
[30] Goh J S Y, Liu Y, Chan K F, Wan C, Teo G, Zhang P, et al. Producing recombinant therapeutic glycoproteins with enhanced sialylation using CHO-gmt4 glycosylation mutant cells. Bioengineered. 2014; 5:269-73.
[31] Gupta R, Matta K L, Neelamegham S. A systematic analysis of acceptor specificity and reaction kinetics of five human α(2,3)sialyltransferases: Product inhibition studies illustrates reaction mechanism for ST3Gal-I. Biochem Biophys Res Commun. 2016; 469:606-12. doi:10.1016/j.bbrc.2015.11.130.
[32] Taniguchi T, Woodward A M, Magnelli P, McColgan N M, Lehoux S, Jacobo S M P, et al. N-Glycosylation affects the stability and barrier function of the MUC16 mucin. J Biol Chem. 2017; 292:11079-90.
[33] Nielsen M I, Stegmayr J, Grant O C, Yang Z, Nilsson U J, Boos I, et al. Galectin binding to cells and glycoproteins with genetically modified glycosylation reveals galectin-glycan specificities in a natural context. J Biol Chem. 2018; 293:20249-62.
[34] Lee P L, Kohler J J, Pfeffer S R. Association of β-1,3-N-acetylglucosaminyltransferase 1 and β-1,4-galactosyltransferase 1, trans-Golgi enzymes involved in coupled poly-N-acetyllactosamine synthesis. Glycobiology. 2009; 19:655-64. doi:10.1093/glycob/cwp035.
[35] Praissman J L, Live D H, Wang S, Ramiah A, Chinoy Z S, Boons G-J, et al. B4GAT1 is the priming enzyme for the LARGE-dependent functional glycosylation of α-dystroglycan. eLife. 2014; 3:e03943. doi:10.7554/eLife.03943.
[36] Kawano S, Hashimoto K, Miyama T, Goto S, Kanehisa M. Prediction of glycan structures from gene expression data based on glycosyltransferase reactions. Bioinforma Oxf Engl. 2005; 21:3976-82.
[37] Puri A, Neelamegham S. Understanding Glycomechanics using Mathematical Modeling: A review of current approaches to simulate cellular glycosylation reaction networks. Ann Biomed Eng. 2012; 40:816-27. doi:10.1007/s10439-011-0464-5.
[38] Lee J, Sundaram S, Shaper N L, Raju T S, Stanley P. Chinese Hamster Ovary (CHO) Cells May Express Six β4-Galactosyltransferases (β4GalTs) CONSEQUENCES OF THE LOSS OF FUNCTIONAL f34GalT-1, β4GalT-6, OR BOTH IN CHO GLYCOSYLATION MUTANTS. J Biol Chem. 2001; 276:13924-34. doi: 10.1074/jbc.M010046200.
[39] Chung C-Y, Yin B, Wang Q, Chuang K-Y, Chu J H, Betenbaugh M J. Assessment of the coordinated role of ST3GAL3, ST3GAL4 and ST3GAL6 on the α2,3 sialylation linkage of mammalian glycoproteins. Biochem Biophys Res Commun. 2015; 463:211-5. doi:10.1016/j.bbrc.2015.05.023.
[40] Chiang A W T, Li S, Spahn P N, Richelle A, Kuo C-C, Samoudi M, et al. Modulating carbohydrate-protein interactions through glycoengineering of monoclonal antibodies to impact cancer physiology. Curr Opin Struct Biol. 2016; 40:104-11. doi: 10.1016/j.sbi.2016.08.008.
[41] Heirendt L, Arreckx S, Pfau T, Mendoza S N, Richelle A, Heinken A, et al. Creation and analysis of biochemical constraint-based models using the COBRA Toolbox v.3.0. Nat Protoc. 2019; 14:639. doi:10.1038/s41596-018-0098-2.
[42] Conn A, Gould N, Toint P. A Globally Convergent Augmented Lagrangian Algorithm for Optimization with General Constraints and Simple Bounds. SIAM J Numer Anal. 1991; 28:545-72. doi:10.1137/0728030.
[43] Lewis R, Shepherd A, Torczon V. Implementing Generating Set Search Methods for Linearly Constrained Minimization. SIAM J Sci Comput. 2007; 29:2507-30. doi: 10.1137/050635432.
[44] García-Ródenas R, Linares L J, López-Gómez J A. A Memetic Chaotic Gravitational Search Algorithm for unconstrained global optimization problems. Appl Soft Comput. 2019; 79:14-29. doi:10.1016/j.asoc.2019.03.011.
[45] Kolda T G, Lewis R M, Torczon V. A generating set direct search augmented Lagrangian algorithm for optimization with a combination of general and linear constraints. 2006.
[46] Deb K, Srivastava S. A genetic algorithm based augmented Lagrangian method for constrained optimization. Comput Optim Appl. 2012; 53:869-902. doi:10.1007/s10589-012-9468-9.
[47] Megchelenbrink W, Huynen M, Marchiori E. optGpSampler: An Improved Tool for Uniformly Sampling the Solution-Space of Genome-Scale Metabolic Networks. PLOS ONE. 2014; 9:e86587. doi:10.1371/journal.pone.0086587.
[48] Hou W, Qiu Y, Hashimoto N, Ching W-K, Aoki-Kinoshita K F. A systematic framework to derive N-glycan biosynthesis process and the automated construction of glycosylation networks. BMC Bioinformatics. 2016; 17:240. doi:10.1186/s12859-016-1094-6.
[49] Krambeck F J, Bennun S V, Narang S, Choi S, Yarema K J, Betenbaugh M J. A mathematical model to derive N-glycan structures and cellular enzyme activities from mass spectrometric data. Glycobiology. 2009; 19:1163-75. doi:10.1093/glycob/cwp081.
[50] Ardèvol A, Rovira C. Reaction Mechanisms in Carbohydrate-Active Enzymes: Glycoside Hydrolases and Glycosyltransferases. Insights from ab Initio Quantum Mechanics/Molecular Mechanics Dynamic Simulations. J Am Chem Soc. 2015; 137:7528-47. doi:10.1021/jacs.5b01156.
[51] Yang Q, Zhang R, Cal H, Wang L-X. Revisiting the substrate specificity of mammalian α1,6-fucosyltransferase reveals that it catalyzes core fucosylation of N-glycans lacking α1,3-arm GlcNAc. J Biol Chem. 2017; 292:14796-803.
[52] Castilho A, Gruber C, Thader A, Oostenbrink C, Pechlaner M, Steinkellner H, et al. Processing of complex N-glycans in IgG Fc-region is affected by core fucosylation. mAbs. 2015; 7:863-70. doi:10.1080/19420862.2015.1053683.
[53] Amann T, Hansen A H, Kol S, Hansen H G, Arnsdorf J, Nallapareddy S, et al. Glyco-engineered CHO cell lines producing alpha-1-antitrypsin and C1 esterase inhibitor with fully humanized N-glycosylation profiles. Metab Eng. 2019; 52:143-52.
[54] Kruschke J K. Bayesian estimation supersedes the t test. J Exp Psychol Gen. 2013; 142:573-603.
[55] Kruschke J K, Liddell T M. The Bayesian New Statistics: Hypothesis testing, estimation, meta-analysis, and power analysis from a Bayesian perspective. Psychon Bull Rev. 2018; 25:178-206. doi:10.3758/s13423-016-1221-4.
[56] Winter N. Matlab Toolbox for Bayesian Estimation. Contribute to NilsWinter/matlab-bayesian-estimation development by creating an account on GitHub. Matlab. 2019. https://github.com/NilsWinter/matlab-bayesian-estimation. Accessed 1 Apr. 2019.
[57] Depaoli S, Clifton J P, Cobb P R. Just Another Gibbs Sampler (JAGS): Flexible Software for MCMC Implementation. J Educ Behav Stat. 2016; 41:628-49. doi: 10.3102/1076998616664876.
[58] Muller P, Parmigiani G, Rice K. FDR and Bayesian Multiple Comparisons Rules. Johns Hopkins Univ Dept Biostat Work Pap. 2006. https://biostats.bepress.com/jhubiostat/paper115.
[59] Beerli P. Comparison of Bayesian and maximum-likelihood inference of population genetic parameters. Bioinformatics. 2006; 22:341-5. doi:10.1093/bioinformatics/bti803.
[60] Guo H-B, Nairn A, Harris K, Randolph M, Alvarez-Manilla G, Moremen K, et al. Loss of expression of N-acetylglucosaminyltransferase Va results in altered gene expression of glycosyltransferases and galectins. FEB S Lett. 2008; 582:527-35. doi: 10.1016/j.febslet.2008.01.015.
[61] Jeong Y T, Choi O, Lim H R, Son Y D, Kim H J, Kim J H. Enhanced sialylation of recombinant erythropoietin in CHO cells by human glycosyltransferase expression. J Microbiol Biotechnol. 2008; 18:1945-52.
[62] Padra, J. T.; Sundh, H.; Jin, C.; Karlsson, N. G.; Sundell, K.; Lindeń, S. K. Aeromonas salmonicida binds differentially to mucins isolated from skin and intestinal regions of Atlantic salmon in an N-acetylneuraminic acid-dependent manner. Infect. Immun. 2014, 82, 5235-5245.
[63] Tarp M A, Clausen H. Mucin-type O-glycosylation and its potential use in drug and vaccine development. Biochim Biophys Acta. 2008 March; 1780(3):546-63
[64] Jin C, Padra J T, Sundell K, Sundh H, Karlsson N G, Linden S K. Atlantic Salmon Carries a Range of Novel O-Glycan Structures Differentially Localized on Skin and Intestinal Mucins. J Proteome Res. 2015 Aug. 7; 14(8):3239-5.1
[65] Joshi H J, Narimatsu Y, Schjoldager K T, Tytgat H L P, Aebi M, Clausen H, Halim A. SnapShot: O-Glycosylation Pathways across Kingdoms. Cell. 2018 Jan. 25; 172(3):632-632.e2.
[66] Liang, C., Chiang, A. W. T., Hansen, A. H., Arnsdorf, J., Schoffelen, S., Sorrentino, J. T., Kellman, B. P., Bao, B., Voldborg, B. G., and Lewis, N. E. (2020). A Markov model of glycosylation elucidates isozyme specificity and glycosyltransferase interactions for glycoengineering. Current Research in Biotechnology 2,22-36.
[67] Quehenberger O, Armando A M, Brown A H, Milne S B, Myers D S, Merrill A H, Bandyopadhyay S, Jones K N, Kelly S, Shaner R L, Sullards C M, Wang E, Murphy R C, Barkley R M, Leiker T J, Raetz C R, Guan Z, Laird G M, Six D A, Russell D W, McDonald J G, Subramaniam S, Fahy E, Dennis E A. Lipidomics reveals a remarkable diversity of lipids in human plasma. J Lipid Res , [Epub ahead of print] (2010)
[68] Han, X. (2016). Lipidomics for studying metabolism. Nature Reviews Endocrinology 12, 668-679.
[69] Lydic, T. A., and Goo, Y.-H. (2018). Lipidomics unveils the complexity of the lipidome in metabolic diseases. Clin Transl Med 7.
[70] Mc Auley, M. T., and Mooney, K. M. (2015). Computationally Modeling Lipid Metabolism and Aging: A Mini-review. Computational and Structural Biotechnology Journal 13,38-46.
[71] Schützhold, V., Hahn, J., Tummler, K., and Klipp, E. (2016). Computational Modeling of Lipid Metabolism in Yeast. Front. Mol. Biosci. 3.
[72] Shih, A. Y., Arkhipov, A., Freddolino, P. L., and Schulten, K. (2006). A coarse grained protein-lipid model with application to lipoprotein particles. J Phys Chem B 110,3674-3684.
[73] Tsouka, S., and Hatzimanikatis, V. (2020). redLips: a comprehensive mechanistic model of the lipid metabolic network of yeast. FEMS Yeast Res 20.

1. A method for determining the biosynthetic basis of a glycosylation pattern or lipid pattern on a cell, glycolipid, tissue, or a protein to be produced by a cell, or an organism to be engineered, comprising:
a. quantifying the impact on the abundance of a glycan or lipid, stemming from enzyme mutation, gene/protein expression changes, or activities of other enzymes to learn enzyme specificity and enzyme interaction rules
b. applying learned enzyme specificity and enzyme interaction rules for glycosylation pattern or lipid pattern to predict an outcome of enzyme mutations or gene/protein expression changes on the glycosylation or lipid pattern on a studied protein, lipid, or cell.

2. The method according to claim 1 where the enzyme is a glycosyltransferase (GT) or a glycosidase or an enzyme in lipid biosynthesis or lipid degradation.

3. The method according to claim 1, wherein the enzyme mutations occur by natural mutations, or non-naturally by modification of the gene sequence or post-translational modification or enzyme activity through cell culture or chemical treatment, or by changing gene/protein expression levels by natural or non-natural means.

4. The method according to claim 1, wherein the mutations or gene/protein expression changes occur on a single enzyme or multiple enzymes.

5. The method according to claim 1, wherein the lipids or glycans are free or are attached to a protein, lipid, tissue, recombinant vaccine, or a cell.

6. The method according to claim 1, wherein the biological source of the glycosylation pattern or lipid pattern is either the same product or a different product from the control product.

7. The method according to claim 1, wherein the method utilizes a Markov model.

8. The method according to claim 1, wherein the method to quantify enzyme mutational effects on reactions catalyzed by other enzymes utilizes Markov transition probabilities.

9. The method according to claim 1, wherein enzyme mutations or gene/protein expression changes are in different enzymes and/or isozymes.

10. The method according to claim 1, wherein the cell is a eukaryotic cell.

11. The method according to claim 1, wherein the glycosylation is N linked glycosylation, O-linked glycosylation, or glycolipid.

12. The method according to claim 1, wherein the tissue is any kind of tissue.

13. The method according to claim 1, wherein the organism is a microbe, a plant, or an animal.

14. A method of producing a protein or lipid or cell or tissue or organism having a desired lipid or glycosylation pattern comprising determining a glycosylation pattern by the method of claim 1 and producing the glycosylated protein or lipid or cell.

15. A glycan or lipid that is free or part of a protein or cell or tissue or organism produced by the method of claim 14.

16. The method of producing a protein or lipid or tissue or organism according to claim 14, wherein the method is conducted in a biopharmaceutical manufacturing facility.

17. A method of treating a subject in need in need thereof, the method comprising administering to the subject a treatment effective amount of the glycosylated protein or lipid or cell or tissue or organism of claim 14.

18. A method of treatment for a biological sample in need comprising administering to the subject a treatment effective amount of the glycosylated protein or lipid or cell of claim 14, wherein the biological sample comprises mammalian cell.

19. The method according to claim 2, where the enzyme is selected from table 1 or table 3.

20. The method according to claim 12, wherein the tissue is selected from skin, pyloric caeca, and proximal intestine.
